(19) United States
(12) Patent Application Publication
(10) Pub. No.: US 2023/0160894 A1
(43) Pub. Date: May 25, 2023

(54) A Diagnostic Device

(71) Applicant: Bio-Diagnostics Limited, Upton-Upon-Severn (GB)

(72) Inventor: Simon Mico, Upton-Upon-Severn (GB)

(21) Appl. No.: 17/922,896

(22) PCT Filed: May 4, 2021
(86) PCT No.: PCT/GB2021/051071

§ 371 Date: Nov. 2, 2022

(30) Foreign Application Priority Data
May 4, 2020 (GB) 2006581.9
Oct. 23, 2020 (GB) 2016894.4

(51) Int. Cl. G01N 33/569; G01N 33/543

(52) U.S. Cl. CPC G01N 33/56983; G01N 33/54386; G01N 2333/165; G01N 2469/20

(57) ABSTRACT

A diagnostic device (1) for detecting a first member of a reporter-analyte pair. The diagnostic device comprises an inlet for receiving a liquid, biological sample and a porous membrane element (10) comprising a detection portion. The detection portion is in liquid communication with the inlet and a second member of the reporter-analyte pair is immobilised on the detection portion. One of the first or second member of the reporter-analyte pair comprises a biological antigen and the other of the first or second member of the reporter-analyte pair comprises an antibody specific for the biological antigen. The biological antigen comprises a spike protein, or a fragment thereof, of COVID-19. The device is for independent detection of the spike protein, or the fragment thereof, or of an antibody specific for the spike protein, or the fragment thereof, in the biological sample.

### FIELD OF THE INVENTION

The present invention relates to a diagnostic device and a kit comprising the diagnostic device and also to a method of testing for the presence of an antibody specific for a biological antigen.

### BACKGROUND OF THE INVENTION

Coronavirus disease 2019 (COVID-19) is an infectious disease caused by severe acute respiratory syndrome coronavirus 2 (SARS-CoV-2). The disease was first identified in December 2019 in Wuhan, the capital of China's Hubei province, and has since spread globally, resulting in the ongoing 2019-20 coronavirus pandemic. As of 1 May 2020, more than 3.27 million cases have been reported across 187 countries and territories, resulting in more than 233,000 deaths. Left unchecked, it is estimated that 40-70% of the global population of approximately 7.8 billion will contract the current form of the virus. With an estimated mortality of 1-3%, 31-164 million lives are at risk.

Repeat testing, via detection of antibodies, and specifically IgG antibodies, for potential immunity and post infection epidemiology data, and also testing for viral antigens, will provide an essential tool to minimise the continued spread of disease, and risk of its mutation into something even more deadly.

COVID-19 is most contagious during the first three days after the onset of symptoms, although spread may be possible before symptoms appear and in later stages of the disease. Since the virus is highly contagious and since the incubation period in an individual can be up to 14 days (and, indeed, certain infected individuals do not present any noticeable symptoms at all) monitoring the progress of the infection within a community is challenging. It is widely accepted that a high degree of testing within communities is desirable in order to identify individuals who are currently infected and to identify individuals who have previously been infected and currently have antibodies specific for the virus.

A wide range of different techniques have been devised for testing for the COVID-19 virus. A typical test for the virus, itself, is a reverse transcription polymerase chain reaction test which detects RNA from the virus and for which results are generally available within a few hours to 2 days. A related test is an isothermal amplification assay.

Another type of test is an antigen test which seeks to detect proteins (in some cases proteins from the surface spikes) from the surface of the virus. The problem with this type of test is that often the amount of antigenic material present in a biological sample is not enough to be readily detected. Based on the sensitivity of similar antigen tests for respiratory diseases such as influenza, it is currently doubtful whether a test of this type could be made sufficiently reliable for the detection of the COVID-19 virus.

A further type of test is a serology test in which antibodies, which are presumed to be specific for epitopes of the COVID-19 virus, are detected in blood samples from an individual. Typically, such tests are either lateral flow immunoassay tests (which are invariably coated with nucleoprotein antigen) or are enzyme-linked immunosorbent assay (ELISA) tests. The problem with lateral flow immunoassay tests is that, although they are suited for domestic use and use by non-medical practitioners, their accuracy and reliability, in particular their sensitivity and specificity, in relation to the detection of the COVID-19 virus is questionable. The inherent design limitations of lateral flow immunoassay test devices and the lack of bonds to hold components together results in the test strips in the devices invariably being loose and able to detach from the correct position in relation to other components. The sample volume of lateral flow amino acid test devices is also very limited due to design limitations and the way in which the molecular elements are stacked in a complex arrangement. Furthermore, a lateral flow immunoassay test can take 20 minutes to complete. The problem with ELISA tests is that it can take 1 to 5 hours to give results and must typically be performed in an equipped laboratory, with skilled staff working in controlled conditions

Another type of test which has hitherto not been applied to the COVID-19 virus is the QuickCard™ test. The QuickCard test has predominantly been applied in the detection of autoimmune diseases. In its typical form, the QuickCard test comprises a rigid casing with an aperture therein and a nitrocellulose membrane located so as to cover the aperture and being supported on a liquid-absorbent pad. The nitrocellulose membrane has a biological antigen immobilised thereon. In use, a liquid, biological sample (typically a blood sample from an individual) is deposited on the nitrocellulose membrane via the aperture in the casing such that any relevant antibodies in the sample bind to the biological antigen. Excess liquid from the sample is then absorbed through the nitrocellulose membrane and into the liquid-absorbent pad. In a subsequent step, a detection reagent is deposited on the nitrocellulose membrane, via the aperture in the casing. The detection reagent comprises a gold particle conjugated to an anti-IgG, anti-IgM or anti-IgA antibody. If the biological sample contained relevant antibodies then the antibody in the detection reagent binds to the relevant antibodies, thus concentrating the gold particles onto the nitrocellulose membrane such that a detectable spot is visible. Thus the presence of the spot is indicative of the presence of relevant antibodies (IgG, IgM or IgA) in the biological sample whereas the absence of the spot is indicative of the absence of relevant antibodies from the biological sample. The presence of antibodies is indicative either of prior infection by a micro-organism (e.g. virus) against which the antibodies have been generated or immunity arising from prior vaccination.

While the QuickCard test is an effective test for many antibodies, it has been found that in certain circumstances, such as the detection of anti-COVID-19 antibodies, the usual configuration of the QuickCard test is not sufficiently sensitive to provide a reliable test.

The QuickCard test was developed in the mid-1990s. Various iterations of the test have been developed over the years. One such variant is disclosed in WO95/19845, the contents of which is hereby incorporated by reference.

The present invention seeks to alleviate one or more of the above problems.

### SUMMARY OF THE INVENTION

The present invention arises from the surprising finding that, by providing a flow control structure between the nitrocellulose membrane (or other porous membrane element) and the casing of the QuickCard test, flow of the liquid, biological sample is better controlled. In particular, the flow control structure limits or prevents the seepage of the liquid sample around the outer surface of the nitrocellulose membrane (or other porous membrane element) and directly to the liquid-absorbent pad, without coming into contact with the biological antigen (or other member of a reporter-analyte pair) immobilised on the nitrocellulose membrane. By avoiding such seepage, the variability between manufactured test kits is significantly reduced making the test kit more reproducible and thus more reliable. In particular, the reliability of the test is such that detection of antibodies specific for the COVID-19 virus in a biological sample is possible.

The present invention also arises from the surprising finding that, when detecting whether an individual has been exposed to the COVID-19 virus (i.e. either through infection or through vaccination), identifying the presence of antibodies against the COVID-19 spike protein and specifically the S1 spike protein is a much more accurate indication of the exposure of the individual to the COVID-19 virus than is identifying the presence of antibodies against other parts of the virus. In particular, the present invention arises from the finding that the nucleoprotein of the COVID-19 virus is not specific to the COVID-19 virus and is also present in other coronaviruses, giving rise to the potential for cross reactive results. Thus, by detecting the presence of antibodies against the spike protein and, specifically, the S1 spike protein, false positive detection events are avoided.

The present invention also arises from the observation that in certain contexts (e.g. where a detection device has to be produced rapidly) the quality of the biological antigen that is provided in a detection device may be sub-optimal. For example, the biological antigen may be contaminated with antigens from the host species, which gives rise to false positive detection events due to reactivity against other antigens of the host species. Thus the present invention arises from the realisation that providing multiple versions of the biological antigen and detecting the presence of antibodies against all versions of the biological antigen such that only the detection of antibodies against all versions is regarded as a positive result gives rise to a more accurate detection device.

The present invention also arises from the realisation that providing a detection device which gives an indication of the quantity of antibody present in a sample can be achieved by providing multiple different concentrations of antigen in the detection device.

The present invention also arises from the recognition that, where only a low volume of biological sample is available for testing in a detection device, funneling the biological sample such that all or almost all of the sample comes into contact with the detection portion on which antigen is located ensures that the biological sample is used to maximum effect and does not bypass the detection portion.

The present invention also arises from the finding that, in a detection device, locating biological antigen on a non-planar membrane facilitates greater control of detection of antibodies in a biological sample. Such greater control includes managing low volumes of biological sample by coalescing the biological sample on the biological antigen; and managing a biological sample which contains visual contaminants by encouraging the contaminants to flow away from the biological antigen.

According to one aspect of the present invention, there is provided a diagnostic device for detecting a first member of a reporter-analyte pair comprising:

a casing comprising a testing portion having an aperture therein, the aperture having an inlet for receiving a liquid, biological sample and an outlet for releasing the liquid, biological sample;

a porous membrane element comprising a detection portion, a second member of the reporter-analyte pair being immobilised on the detection portion, the outlet being in liquid communication with the detection portion via a detection flow path; and

a flow control structure for limiting or preventing the flow of the liquid, biological sample from the outlet along a bypass flow path, which bypasses the detection portion, instead of the detection flow path.

Conveniently, the flow control structure comprises a liquid-tight seal present between the testing portion and the porous membrane element, which prevents the liquid, biological sample from flowing along the bypass flow path which bypasses the detection portion, preferably wherein the liquid-tight seal surrounds the aperture.

Preferably, the liquid-tight seal is an ultrasonic weld or other controlled seal between the testing portion and the porous membrane element.

Advantageously, the casing and the liquid-tight seal form a liquid-tight chamber around the porous membrane element from which only the detection portion of the porous membrane element is exposed from the chamber.

Alternatively, the flow control structure comprises an arrangement of gates or obstacles which are present in the bypass flow path and which limit the liquid, biological sample from flowing along the bypass flow path which bypasses the detection portion.

Conveniently, the porous membrane element is located adjacent to the testing portion such that the detection portion is visible through the inlet of the aperture.

Preferably, the diagnostic device further comprises a liquid-absorbent pad in liquid-communication with the detection portion of the porous membrane element, the detection flow path being from the outlet of the aperture through the detection portion and into the liquid-absorbent pad.

Advantageously, the bypass flow path is from the outlet of the aperture to the liquid-absorbent pad, without passing through the detection portion of the porous membrane element.

Conveniently, the liquid-absorbent pad comprises an opening therein, and the porous membrane element is located between the liquid-absorbent pad and the aperture in the testing portion, the opening in the liquid-absorbent pad being aligned with the aperture in the testing portion.

Preferably, the porous membrane element comprises a nitro-cellulose membrane.

Advantageously, the second member of the reporter-analyte pair comprises a biological antigen and the first member of the reporter-analyte pair comprises an antibody specific for the biological antigen.

Conveniently, the biological antigen comprises a Coronavirus protein or fragment thereof.

Preferably, the coronavirus protein is a Covid-19 protein, preferably a COVID-19 S1 spike protein or fragment thereof. Advantageously, the biological antigen does not comprise the Covid-19 nucleoprotein, or a fragment thereof, of COVID-19 but conveniently does comprise the COVID-19 S2 protein (which follows the RBD portion of the S1 protein).

Advantageously, the coronavirus protein or fragment thereof is a polypeptide comprising a sequence of at least 8, 10, 12, 14, 16, 18, 20, 30, 100, 200 or 300 amino acids from an amino acid sequence having at least 70%, 80%, 90%, 95%, 99% or 100% sequence identity to the sequence represented in FIG. 10 (SEQ ID NO: 4).

Alternatively, the first member of the reporter-analyte pair comprises a biological antigen and the second member of the reporter-analyte pair comprises an antibody specific for the biological antigen.

Conveniently, the biological antigen comprises a COVID-19 protein or fragment thereof.

Preferably, the casing comprises at least one securing structure for locating the porous membrane element in a position covering the aperture.

Advantageously, the porous membrane element further comprises a reference element for indicating a level of the first member of the reporter-analyte pair in the liquid, biological sample, preferably wherein the reference element is a plurality of reference elements.

According to a second aspect of the present invention, there is provided a kit comprising a diagnostic device according to the invention and a detection reagent, the detection reagent comprising a molecular conjugate which comprises a detectable moiety bound to a moiety capable of binding the first member of the reporter-analyte pair.

Conveniently, the moiety capable of binding the first member of the reporter-analyte pair is an antibody.

Preferably, the molecular conjugate comprises a gold particle conjugated to an anti-IgG or anti-IgM antibody.

According to a third aspect of the present invention, there is provided a method of testing for the presence of a first member of a reporter-analyte pair in a liquid, biological sample, comprising the steps of:

I) providing a diagnostic device according to the present invention;

II) depositing the biological sample onto the porous membrane element via the aperture such that the biological sample contacts the second member of the reporter-analyte pair immobilised on the porous membrane element and such that first member of the reporter-analyte pair interacts with the second member of the reporter-analyte pair;

III) depositing a detection reagent onto the porous membrane element via the aperture such that the detection agent contacts the first member of the reporter-analyte pair, the detection reagent providing a detectable signal when contacting the first member of the reporter-analyte pair.

Conveniently, the detection reagent comprises a detectable moiety bound to a moiety capable of binding the first member of the reporter-analyte pair.

According to a fourth aspect of the present invention, there is provided a diagnostic device for detecting a first member of a reporter-analyte pair in a biological sample comprising:
a porous membrane element comprising a detection portion, a second member of the reporter-analyte pair being immobilised on the detection portion, wherein one of the first or second member of the reporter-analyte pair comprises a biological antigen and the other of the first or second member of the reporter-analyte pair comprises an antibody specific for the biological antigen, and

wherein the biological antigen comprises a spike protein, or a fragment thereof, of COVID-19 and wherein the device is for independent detection of the spike protein, or the fragment thereof, or of an antibody specific for the spike protein, or the fragment thereof, in the biological sample.

Preferably, the second member of the reporter-analyte pair is immobilisable on the detection portion.

Conveniently, the diagnostic device comprises an inlet for receiving a liquid, biological sample, wherein the inlet and the detection portion are in liquid communication.

Preferably, the spike protein or the fragment thereof is a COVID-19 S1 spike protein or a fragment thereof, optionally the COVID-19 S1 spike protein receptor binding domain.

Conveniently, the COVID-19 S1 spike protein or the fragment thereof is a polypeptide comprising a sequence of at least 8, 10, 12, 14, 16, 18, 20, 30, 100, 200 or 300 amino acids from an amino acid sequence having at least 70%, 80%, 90%, 95%, 99% or 100% sequence identity to the sequence represented in FIG. 10 (SEQ ID NO: 4).

Advantageously, the COVID-19 S1 spike protein or the fragment thereof comprises the COVID-19 S1 spike protein receptor binding domain having the sequence of SEQ ID NO: 3.

Advantageously, the reporter-analyte pair is a plurality of reporter-analyte pairs, wherein one of the first or second member of each reporter-analyte pair comprises a biological antigen and the other of the first or second member of each reporter-analyte pair comprises an antibody specific for the biological antigen, wherein the biological antigen of each reporter-analyte pair is a different biological antigen.

Conveniently, the plurality of reporter-analyte pairs comprises a first, a second and optionally a third reporter-analyte pair,

wherein the biological antigen of the first reporter-analyte pair is a COVID-19 S1 spike protein or a fragment thereof, and

wherein the biological antigen of the second reporter-analyte pair is a COVID-19 S2 spike protein or a fragment thereof, and optionally

wherein the biological antigen of the third reporter-analyte pair is a COVID-19 nucleoprotein or a fragment thereof.

Preferably, the COVID-19 S2 spike protein or the fragment thereof is a polypeptide comprising a sequence of at least 8, 10, 12, 14, 16, 18, 20, 30, 100, 200 or 300 amino acids from an amino acid sequence having at least 70%, 80%, 90%, 95%, 99% or 100% sequence identity to the sequence represented in FIG. 11A (SEQ ID NO: 5) or FIG. 11B (SEQ ID NO: 6).

Advantageously, the COVID-19 S2 spike protein or the fragment thereof comprises a sequence in which the C-terminal six amino acids are deleted from the sequence represented in FIG. 11A (SEQ ID NO: 5) or FIG. 11B (SEQ ID NO: 6).

Preferably, the COVID-19 S2 spike protein or the fragment thereof comprises a sequence in which the C-terminal sixty-two amino acids are deleted from the sequence represented in FIG. 11A (SEQ ID NO: 5) or FIG. 11B (SEQ ID NO: 6).

Conveniently, the COVID-19 S1 spike protein or the fragment thereof and the COVID-19 S2 spike protein or the fragment thereof are comprised within a single polypeptide chain, the single polypeptide chain being immobilised on the detection portion.

Preferably, the single polypeptide chain is immobilisable on the detection portion.

Preferably, the COVID-19 nucleoprotein or the fragment thereof comprises the sequence represented in FIG. 12 (SEQ ID NO: 7).

Advantageously, the porous membrane element further comprises a reference element for indicating a level of the first member of the or each reporter-analyte pair in the liquid, biological sample, preferably wherein the reference element is a plurality of reference elements.

According to a fifth aspect of the present invention, there is provided a kit comprising a diagnostic device according to any one of the preceding claims and a detection reagent, the detection reagent comprising a molecular conjugate which comprises a detectable moiety bound to a moiety capable of binding the first member of the reporter-analyte pair.

Conveniently, the moiety capable of binding the first member of the reporter-analyte pair is an antibody.

Advantageously, the molecular conjugate comprises a gold particle conjugated to an anti-IgG or anti-IgM antibody.

According to a sixth aspect of the present invention, there is provided a method of testing for the presence of a first member of a reporter-analyte pair in a liquid, biological sample, comprising the steps of:

I) providing a diagnostic device according to the present invention;

II) depositing the biological sample onto the porous membrane element such that the biological sample contacts the second member of the reporter-analyte pair immobilised on the porous membrane element and such that first member of the reporter-analyte pair interacts with the second member of the reporter-analyte pair, wherein an interaction between the first member of the reporter-analyte pair and the second member of the reporter-analyte pair is detectable.

Advantageously, the second member of the reporter-analyte pair is immobilisable on the porous membrane element.

Conveniently, the method further comprises the step of:

III) depositing a detection reagent onto the porous membrane element such that the detection reagent contacts the first member of the reporter-analyte pair, the detection reagent providing a detectable signal when contacting the first member of the reporter-analyte pair.

Advantageously, the detection reagent comprises a detectable moiety bound to a moiety capable of binding the first member of the reporter-analyte pair.

Preferably, the second member of the reporter-analyte pair comprises a biological antigen, the biological antigen comprising a spike protein, or a fragment thereof, of COVID-19 and wherein the first member of the reporter-analyte pair comprises an antibody specific for the spike protein, or the fragment thereof, of COVID-19.

Conveniently, the reporter-analyte pair is a first and/or a second reporter-analyte pair, wherein the second member of the first reporter-analyte pair is a COVID-19 S1 spike protein or a fragment thereof and the first member of the first reporter-analyte pair is an antibody specific for the COVID-19 S1 spike protein or a fragment thereof; and/or wherein the second member of the second reporter-analyte pair is a COVID-19 S2 spike protein or a fragment thereof and the first member of the second reporter-analyte pair is an antibody specific for the COVID-19 S2 spike protein or a fragment thereof, and wherein the presence of an antibody specific for the COVID-19 S1 spike protein or the fragment thereof and/or the COVID-19 S2 spike protein or the fragment thereof in the biological sample indicates current or prior infection by or vaccination against COVID-19.

Alternatively, the reporter-analyte pair is a first, a second and a third reporter-analyte pair,

wherein the second member of the first reporter-analyte pair is a COVID-19 S1 spike protein or a fragment thereof and the first member of the first reporter-analyte pair is an antibody specific for the COVID-19 S1 spike protein or a fragment thereof;

wherein the second member of the second reporter-analyte pair is a COVID-19 S2 spike protein or a fragment thereof and the first member of the second reporter-analyte pair is an antibody specific for the COVID-19 S2 spike protein or a fragment thereof; and wherein the second member of the third reporter-analyte pair is a COVID-19 nucleoprotein or a fragment thereof and the first member of the third reporter-analyte pair is an antibody specific for the COVID-19 nucleoprotein or a fragment thereof, and wherein the presence of an antibody specific for the COVID-19 nucleoprotein, or the fragment thereof, and the absence of an antibody specific for the COVID-19 S1 spike protein, or the fragment thereof, and the absence of an antibody specific for the COVID-19 S2 spike protein, or the fragment thereof, indicates current or prior infection by or vaccination against a beta group coronavirus other than COVID-19.

According to a seventh aspect of the present invention, there is provided a diagnostic device for detecting a first member of a reporter-analyte pair comprising:
a porous membrane element comprising a detection portion, wherein the detection portion comprises a second member of the reporter-analyte pair and a variant of the second member of the reporter-analyte pair immobilised thereon, and
wherein an interaction between the first member of the reporter-analyte pair and the second member of the reporter-analyte pair is separately detectable from an interaction between the first member of the reporter-analyte pair and the variant of the second member of the reporter-analyte pair.

Preferably, the diagnostic device comprises an inlet for receiving a liquid, biological sample, wherein the inlet is in liquid communication with the detection portion.

Advantageously, the second member of the reporter-analyte pair is immobilised on the detection portion at a discrete site from the variant of the second member of the reporter-analyte pair.

Preferably, a control is immobilised on the detection portion.

Advantageously, the second member of the reporter-analyte pair and/or the variant of the second member of the reporter-analyte pair and/or the control are immobilisable on the detection portion.

Conveniently, the control is positioned between the second member of the reporter-analyte pair and the variant of the second member of the reporter-analyte pair.

Preferably, the second member of the reporter-analyte pair comprises a biological antigen and the first member of the reporter-analyte pair comprises an antibody specific for the biological antigen.

Conveniently, the biological antigen is a protein, a polypeptide or a fragment thereof.

Advantageously, the second member of the reporter-analyte pair and the variant of the second member of the reporter-analyte pair are produced in a first and a second host cell respectively, wherein the first and the second host cell are each from a different species.

Preferably, the second member of the reporter-analyte pair and the variant of the second member of the reporter-analyte pair comprise (a) and (b) below respectively:

(a) a biological antigen comprising a COVID-19 S1 spike protein or a fragment thereof, preferably wherein the COVID-19 S1 spike protein or the fragment thereof is a polypeptide comprising a sequence of at least 8, 10, 12, 14, 16, 18, 20, 30, 100, 200 or 300 amino acids from an amino acid sequence having at least 70%, 80%, 90%, 95%, 99% or 100% sequence identity to the sequence of SEQ ID NO: 4 (as represented in FIG. 10); and

(b) a biological antigen comprising a sequence having at least 70% sequence identity and less than 100% sequence identity to (a).

Conveniently, the variant of the second member of the reporter-analyte pair is a plurality of variants.

Advantageously, the porous membrane element further comprises a reference element for indicating a level of the first member of the reporter-analyte pair in the liquid, biological sample, preferably wherein the reference element is a plurality of reference elements.

According to an eighth aspect of the present invention, there is provided a diagnostic device for detecting a first member of a reporter-analyte pair comprising:
a porous membrane element comprising a detection portion, wherein the detection portion comprises a second member of the reporter-analyte pair immobilised thereon at a plurality of different concentrations, and
wherein the plurality of different concentrations of the second member of the report-analyte pair extend between a first point on the detection portion, at which the concentration of the second member of the reporter-analyte pair is highest, through to a second point on the detection portion, at which the concentration of the second member of the reporter-analyte pair is lowest and at sequential concentrations therebetween.

Preferably, the diagnostic device comprises an inlet for receiving a liquid, biological sample, wherein the inlet is in liquid communication with the detection portion.

Conveniently, the plurality of different concentrations of the second member of the reporter-analyte pair extend between the first point and the second point in a substantially continuous gradation of their respective concentrations.

Alternatively, the plurality of different concentrations of the second member of the reporter-analyte pair extend between the first point and the second point at a series of discrete sites.

Advantageously, the diagnostic device further comprises a control.

Advantageously, the porous membrane element further comprises a reference element for indicating a level of the first member of the reporter-analyte pair in the liquid, biological sample, preferably wherein the reference element is a plurality of reference elements.

According to a ninth aspect of the present invention, there is provided a diagnostic device for detecting a first member of a reporter-analyte pair comprising:

a casing comprising a testing portion having an aperture therein, the testing portion having an inlet for receiving a liquid, biological sample and an outlet for releasing the liquid, biological sample;

a membrane element having a detection portion on which a second member of the reporter-analyte pair is immobilised, the outlet being in liquid communication with the detection portion via a detection flow path;

the outlet of the testing portion contacting the membrane element and defining a visible portion of the membrane element, the visible portion of the membrane element comprising the detection portion,

and wherein the surface area of the detection portion is at least 60% but less than 100% of the surface area of the visible portion.

Preferably, the membrane element is a porous membrane element.

Advantageously, the surface area of the detection portion is between 90% and 95% of the surface area of the visible portion.

Conveniently, the testing portion tapers inwardly from the inlet to the outlet.

Advantageously, the testing portion tapers along a variable gradient from the inlet to the outlet such that the testing portion tapers inwardly along a first gradient adjacent to the inlet and along a second gradient adjacent to the outlet, the second gradient being steeper than the first gradient.

According to a tenth aspect of the present invention, there is provided a diagnostic device for detecting a first member of a reporter-analyte pair comprising:

a casing comprising a testing portion having an aperture therein, the testing portion having an inlet for receiving a liquid, biological sample and an outlet for releasing the liquid, biological sample;

a porous membrane element having a detection portion on which a second member of the reporter-analyte pair is immobilised, the outlet being in liquid communication with the detection portion via a detection flow path;

the outlet of the testing portion contacting the membrane element and defining a visible portion of the membrane element, the visible portion of the membrane element comprising the detection portion, wherein the detection portion is non-planar.

Conveniently, the detection portion is concave.

Alternatively, the detection portion is convex.

Preferably, in a diagnostic device of the present invention, the second member of the reporter-analyte pair comprises a biological antigen and the first member of the reporter-analyte pair comprises an antibody specific for the biological antigen.

Conveniently, the biological antigen comprises a spike protein, or a fragment thereof, of COVID-19.

Advantageously, the spike protein comprises a COVID-19 S1 spike protein or a fragment thereof.

Conveniently, the biological antigen is a plurality of biological antigens and comprises a first and a second biological antigen and optionally a third biological antigen, wherein the first biological antigen comprises the COVID-19 S1 spike protein, or a fragment thereof, the second biological antigen comprises the COVID-19 S2 spike protein, or a fragment thereof, and the optional third biological antigen comprises the COVID-19 nucleoprotein, or a fragment thereof.

According to an eleventh aspect of the present invention, there is provided a diagnostic device for detecting a first member of a reporter-analyte pair or a first member of each of a plurality of reporter-analyte pairs comprising:
an inlet for receiving a liquid, biological sample; and
a porous membrane element comprising a detection portion, the detection portion being in liquid communication with the inlet and a second member of the or each reporter-analyte pair being immobilisable on the detection portion,
wherein one of the first or second member of the or each reporter-analyte pair comprises a biological antigen and the other of the first or second member of the or each reporter-analyte pair comprises an antibody specific for the biological antigen,
wherein the biological antigen comprises a COVID-19 S1 spike protein or a fragment thereof comprising a sequence in which one or more amino acids are substituted in a sequence consisting of the N-terminal 100, 200, 300, 400, 500 or 600 amino acids of the sequence of SEQ ID NO: 4 (as represented in FIG. 10), and
wherein the device is for independent detection of the COVID-19 S1 spike protein, or the fragment thereof, in which one or more amino acids has been substituted, or of an antibody specific for the COVID-19 S1 spike protein, or the fragment thereof, in which one or more amino acids has been substituted, in the biological sample.

Advantageously, the porous membrane element further comprises a reference element for indicating a level of the first member of the reporter-analyte pair in the liquid, biological sample, preferably wherein the reference element is a plurality of reference elements.

The present invention encompasses any combination of the aforementioned aspects and embodiments.

The term “polypeptide” as used herein, refers to a polymer of amino acid residues. The term also applies to amino acid polymers in which one or more amino acid residues is a modified residue, or a non-naturally occurring residue, such as an artificial chemical mimetic of a corresponding naturally-occurring amino acid, as well as to naturally occurring amino acid polymers.

The percentage “sequence identity” between two sequences may be determined using the BLASTP algorithm version 2.2.2 (Altschul, Stephen F., Thomas L. Madden, Alejandro A. Schäffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402) using default parameters. In particular, the BLAST algorithm can be accessed on the internet using the URL https://blast.ncbi.nlm.nih.gov/Blast.cgi.

It is preferred that the percentage “sequence identity” between two sequences is determined using EMBOSS Needle Pairwise Sequence Alignment (Rice et al., Trends Genet. 2000 June; 16(6):276-7; Nucleic Acids Res. 2019 Jul. 2; 47(W1):W636-W641) using default parameters. In particular, EMBOSS Needle can be accessed on the internet using the URL: https://www.ebi.ac.uk/Tools/psa/emboss_needle/.

The term “biological antigen” as used herein refers to a molecule capable of eliciting an immune response in an individual. In one embodiment, the “biological antigen” is a protein, a polypeptide or a fragment thereof. In one embodiment, the “biological antigen” comprises a Coronavirus protein or fragment thereof. In a preferred embodiment, the “biological antigen” comprises a COVID-19 protein or a fragment thereof. In some embodiments, the biological antigen comprises a spike protein, or fragment thereof, of COVID-19, the amino acid sequence of which is shown in FIG. 9 (SEQ ID NO: 2). In one embodiment, the spike protein comprises the S1 spike protein, or a fragment thereof, depicted in FIG. 10 (SEQ ID NO: 4). An exemplary fragment of the S1 spike protein is the receptor binding domain which is shown underlined in FIG. 10 (SEQ ID NO: 3). In one embodiment, the spike protein comprises the S2 spike protein, or a fragment thereof, depicted in FIG. 11A (SEQ ID NO: 5) or FIG. 11B (SEQ ID NO: 6).

It is to be understood that the terms “COVID-19” and “SARS-CoV-2” are used interchangeably within the present specification. That is to say, a “COVID-19” protein or fragment thereof refers to a “SARS-CoV-2” protein or fragment thereof.

The term “spike protein, or a fragment thereof, of COVID-19” as used herein refers to a protein comprising the sequence set forth in FIG. 9 (SEQ ID NO: 2) or to a fragment thereof, or to a protein comprising a sequence with at least 70%, 80%, 90%, 95% or 99% sequence identity to the sequence set forth in FIG. 9 (SEQ ID NO: 2) or to a fragment thereof. In one embodiment, the “spike protein, or a fragment thereof, of COVID-19” is a COVID-19 S1 spike protein or a fragment thereof and/or a COVID-19 S2 spike protein or a fragment thereof.

The term “COVID-19 S1 spike protein or a fragment thereof” as used herein refers to a protein comprising the sequence set forth in FIG. 10 (SEQ ID NO: 4) or to a fragment thereof, or to a protein comprising a sequence with at least 70%, 80%, 90%, 95% or 99% sequence identity to the sequence set forth in FIG. 10 (SEQ ID NO: 4) or to a fragment thereof.

The term “COVID-19 S1 spike protein receptor binding domain” as used herein refers to a domain comprising the sequence shown underlined in FIG. 10 (SEQ ID NO: 3) or to a domain comprising a sequence with at least 70%, 80%, 90%, 95% or 99% sequence identity to the sequence shown underlined in FIG. 10 (SEQ ID NO: 3). The receptor binding domain corresponds to amino acid positions 319 to 541 in the sequence of SEQ ID NO: 4. The receptor binding domain comprises a “receptor-binding motif” (RBM) corresponding to amino acid positions 437 to 508 in the sequence of SEQ ID NO: 4. In one embodiment, the fragment of the COVID-19 S1 spike protein comprises or consists of the receptor binding domain (SEQ ID NO: 3) or the receptor binding motif (amino acid positions 437 to 508 in the sequence of SEQ ID NO: 4).

The term “COVID-19 S2 spike protein or a fragment thereof” as used herein refers to a protein comprising a sequence set forth in FIG. 11A (SEQ ID NO: 5) or FIG. 11B (SEQ ID NO: 6) or to a fragment of either, or to a protein comprising a sequence with at least 70%, 80%, 90%, 95% or 99% sequence identity to a sequence set forth in FIG. 11A (SEQ ID NO: 5) or FIG. 11B (SEQ ID NO: 6) or to a fragment of either.

The term “COVID-19 nucleoprotein or a fragment thereof” as used herein refers to a protein comprising the sequence set forth in FIG. 12 (SEQ ID NO: 7) or to a fragment thereof, or to a protein comprising a sequence with at least 70%, 80%, 90%, 95% or 99% sequence identity to the sequence set forth in FIG. 12 (SEQ ID NO: 7) or to a fragment thereof.

The term “independent detection of the spike protein, or the fragment thereof, or of an antibody specific for the spike protein, or the fragment thereof” as used herein refers to detecting specifically a spike protein, or a fragment thereof, of COVID-19 in a biological sample or to detecting an antibody specific for the spike protein, or the fragment thereof, of COVID-19 in a biological sample. Thus the detection of the spike protein, fragment or antibody (as the first member of a reporter-analyte pair) is separate from the detection of other biological antigens or antibodies thereto. In a preferred embodiment, the detection is separate from the detection of a nucleoprotein, or a fragment thereof, of COVID-19 or an antibody specific for the nucleoprotein or the fragment thereof in the biological sample. In one embodiment in which the spike protein or the fragment thereof is immobilised as the second member of a reporter-analyte pair on the detection portion of the diagnostic device, the spike protein or the fragment thereof is in isolation from the nucleoprotein or the fragment thereof. In one embodiment, “in isolation” refers to an absence of the nucleoprotein or the fragment thereof immobilised on the detection portion or to the separate positioning of these elements in relation to the spike protein or the fragment thereof on the detection portion. In such embodiments, the presence or absence, in the biological sample, of an antibody specific for the spike protein or the fragment thereof is detectable, in a separate manner, from the presence or absence of an antibody specific for the nucleoprotein or the fragment thereof. It is to be understood that the above description is with reference to the spike protein, nucleoprotein and fragments thereof as the second member of their respective reporter-analyte pairs but it is also applicable to these elements as the first member of their respective reporter-analyte pairs. In one embodiment, a label is used for independent detection. In a preferred embodiment, the spike protein, or the fragment thereof, of COVID-19 is a COVID-19 S1 spike protein or a fragment thereof.

The term “beta group coronavirus” or “betacoronavirus” as used herein refers to one of four genera (Alpha-, Beta-, Gamma-, and Delta-) of coronaviruses. The beta group coronaviruses comprise enveloped, positive-strand RNA viruses and are capable of infecting humans and mammals. Beta group coronaviruses which are capable of infecting humans include: HCoV-OC43, HCoV-HKU1, SARS-CoV, MERS-CoV and SARS-CoV-2 (COVID-19).

The term “separately detectable” as used herein refers to determining the presence or absence of an interaction between the first and second members of respective reporter-analyte pairs in an independent manner. In one embodiment, “separately detectable” as used herein refers to determining the presence or absence of an interaction between a first member and a second member of a reporter-analyte pair whilst also determining the presence or absence of an interaction between a first member and a variant of the second member of the reporter-analyte pair. In one embodiment, the positioning of each of the second member and variant thereof on a detection portion at a discrete site enables the interactions to be “separately detectable”. In one embodiment, a label is employed for the interactions to be “separately detectable”.

The term “discrete site” as used herein refers to a first location that is distinct and/or spatially separate from a second location. In one embodiment, the first and second locations are positioned on a detection portion.

The term “control” as used herein refers to a comparison against which the readout from the diagnostic device can be evaluated. In one embodiment, the control is a negative control. In an alternative embodiment, the control is a positive control. In one embodiment, the positive control is an anti-IgG antibody. Such a positive control demonstrates that an IgG antibody is present in the biological sample. It is to be understood that the anti-IgG antibody used as the control corresponds to the species from which the biological sample is taken. For example, in one embodiment, the biological sample is taken from a human and the control is an anti-human IgG antibody.

In an alternative embodiment, the biological sample is taken from a dog and the control is an anti-canine IgG antibody.

The term “immobilisable” as used herein refers to capture of the second member of the reporter-analyte pair on the porous membrane element. In one embodiment, the second member is immobilisable on the porous membrane element regardless of the presence or absence of the first member of the reporter-analyte pair. In other embodiments, the second member is immobilisable on the porous membrane element in the presence of the first member of the reporter-analyte pair. The term “immobilised” as used herein encompasses both direct and indirect capture of the second member of the reporter-analyte pair on the porous membrane element. In one embodiment, the second member is immobilised directly on the porous membrane element (i.e. binds directly thereto). In an alternative embodiment, the second member is captured on the porous membrane element indirectly, such as via a linking moiety.

The term “reference element” as used herein refers to an element suitable for indicating a level of the first member of the reporter-analyte pair in the liquid, biological sample. In one embodiment, the reference element comprises a moiety that is capable of a specific interaction with the binding moiety of the molecular conjugate. In one embodiment, the moiety capable of the specific interaction is immobilised on the porous membrane element. In a preferred embodiment, the moiety capable of the specific interaction is an antibody, more preferably an IgG or IgM antibody. In one embodiment, the reference element comprises a plurality of reference elements. In a preferred embodiment, each of the plurality of reference elements comprises a sequential concentration of the moiety capable of the specific interaction. In one embodiment, a detectable signal produced at the reference element is compared with that produced at a detection portion on the porous membrane element and the comparison provides an indication of the level of the first member of the reporter-analyte pair in the liquid, biological sample.

In a preferred embodiment, the membrane element is a porous membrane element. In some embodiments, the membrane element is an absorbent element. In one embodiment, the membrane element is non-porous.

### FIGURES

Embodiments of the invention will now be described with reference to the following figures in which:

FIG. 1 is a perspective view of a diagnostic device in accordance with one embodiment of the present invention in a semi-constructed form;

FIG. 2 is a perspective view of a diagnostic device in accordance with another embodiment of the present invention in a semi-constructed form;

FIG. 3 is a perspective view of a diagnostic device in accordance with one embodiment of the present invention in constructed form;

FIG. 4 is a cross-sectional view of a diagnostic device in accordance with a further embodiment of the present invention;

FIG. 5 is a perspective view of a diagnostic device in accordance with another embodiment of the present invention in a semi-constructed form;

FIG. 6 is a perspective view of a diagnostic device in accordance with another embodiment of the present invention in a semi-constructed form;

FIG. 7 is a perspective view of a diagnostic device in accordance with another embodiment of the present invention in a semi-constructed form;

FIG. 8 is a perspective view of a diagnostic device in accordance with another embodiment of the present invention in constructed form;

FIG. 9 is the amino acid sequence of the 2019-nCov spike protein (1273 amino acids; SEQ ID NO: 2), in which the S1 protein fragment is shown in underline and the predicted cleavage sites are shown in italics (SEQ ID NO: 1);

FIG. 10 is the amino acid sequence of the S1 protein fragment of the 2019-nCov spike protein (SEQ ID NO: 4), in which the amino acid sequence of the receptor binding domain is shown underlined (SEQ ID NO: 3);

FIG. 11A is the amino acid sequence of the S2 subunit (588 amino acids; SEQ ID NO: 5) of the 2019-nCov spike protein, which corresponds to amino acid positions 686 to 1273 of the complete spike protein sequence. FIG. 11B is the amino acid sequence of the S2′ subunit (458 amino acids; SEQ ID NO: 6) of the 2019-nCov spike protein, which corresponds to amino acid positions 816 to 1273 of the complete spike protein sequence;

FIG. 12 is the amino acid sequence of the 2019-nCov nucleoprotein (419 amino acids; SEQ ID NO: 7);

FIG. 13 is a plan view of a testing portion of a diagnostic device in accordance with a further embodiment of the present invention;

FIG. 14 is a plan view of a diagnostic device in accordance with a further embodiment of the present invention;

FIG. 15 is a cross-sectional view of a diagnostic device in accordance with a further embodiment of the present invention;

FIG. 16 is a plan view of the diagnostic device of the embodiment of FIG. 15;

FIG. 17 is a cross-sectional view of a diagnostic device in accordance with a further embodiment of the present invention; and

FIG. 18 is a cross-sectional view of a diagnostic device in accordance with a further embodiment of the present invention;

FIG. 19 is a schematic representation of the interactions between various components in accordance with embodiments of the present invention;

FIG. 20A to 20E are plan views of diagnostic devices and portions thereof in accordance with further embodiments of the present invention;

FIGS. 21A and 21B are plan views of diagnostic devices in accordance with further embodiments of the present invention.

### DETAILED DESCRIPTION

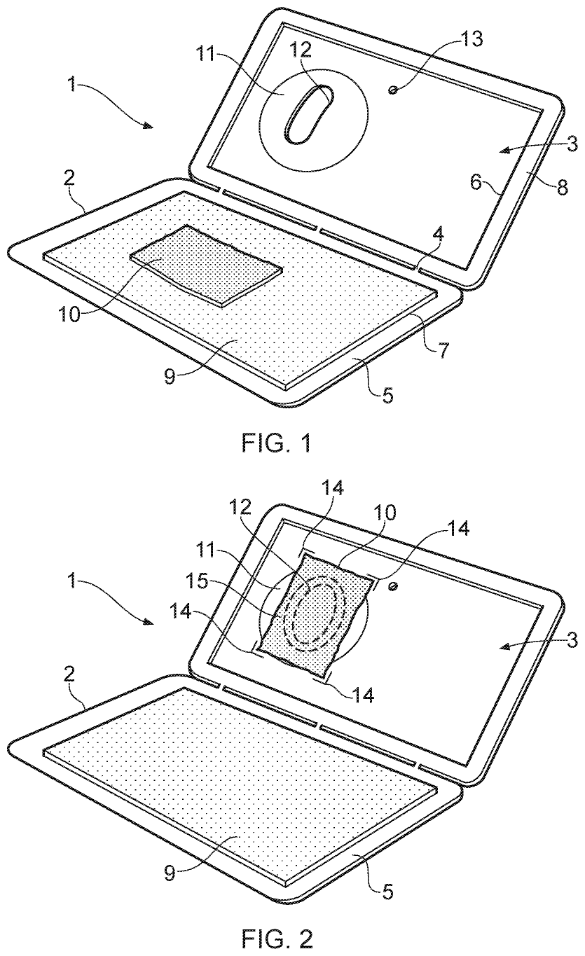

One embodiment of the present invention will now be described with reference to FIG. 1. A diagnostic device 1 comprises a casing which is composed of a rectangular base element 2 which can be connected to a similarly-sized rectangular cover element 3. In alternative embodiments, the base element 2 and the cover element 3 are of different shapes such as being round, oval, square et cetera. In this embodiment, the base element 2 and the cover element 3 are connected by way of a hinge 4 along their respective long sides; however, in alternative embodiments, the hinge 4 is omitted and the base element 2 and the cover element 3 are separate components prior to construction. Each of the base element 2 and the cover element 3 has a respective rectangular tray, 5, 6, defined by a respective upstanding, peripheral lip 7, 8. In this embodiment, the casing is made of a rigid plastics material; however, in alternative embodiments, the casing is made from an alternative material (e.g coated cardboard) which is impervious for sufficient time for the test to be run (e.g at least 20 minutes).

Located within the rectangular tray 5 of the base element 2, there is provided a liquid-absorbent pad 9. The liquid-absorbent pad 9 is rectangular in shape and substantially fills the rectangular tray 5 so as to abut the inner side of the upstanding, peripheral lip 7 within the base element 2. In this embodiment, the liquid-absorbent pad 9 is made from a cotton wadding material overlaid with a porous card such as a blotting paper layer. However, in alternative embodiments, the liquid-absorbent pad is made another absorbent material such as a surface blotting type paper which facilitates adapting the flow rate and analyte contact time for the device.

Positioned on the liquid-absorbent pad 9 is a rectangular nitrocellulose membrane element 10. The pore size of the nitrocellulose membrane element 10 is selected depending upon the target for the test but a pore size in the range of 20 to 100 μm is typical. Immobilised on the nitrocellulose membrane element 10 are a plurality of biological antigens (not shown).

In this embodiment a single liquid-absorbent pad 9, is provided. However, in alternative embodiments, multiple liquid-absorbent pads 9 are provided, positioned such that they are in liquid communication with the nitrocellulose membrane element 10.

Located on the rectangular tray 6 of the cover element 3 is a testing portion 11. The testing portion 11 comprises a circular dimple in the cover element 3 such that the dimple is convex towards the nitrocellulose membrane element 10 as the cover element 3 is moved about the hinge 4 towards the base element 2. In the centre of the circular dimple is a lozenge-shaped aperture 12. In alternative embodiments, the aperture is an alternative shape such as being rectangular, circular, square et cetera.

The aperture 12 and the nitrocellulose membrane element 10 are positioned and sized with respect to each other such that during construction of the diagnostic device 1, when the cover element 3 is swung over the base element 2 about the hinge 4 such that the respective upstanding-peripheral lips 7, 8 mutually engage, the aperture 12 is aligned with the nitrocellulose membrane element 10 and the nitrocellulose membrane element 10 completely covers the aperture 12.

A release hole 13 is also provided through the cover element 3. It is to be understood that the release hole 13 is sufficiently small that surface tension prevents liquids from escaping through it while permitting the escape of gases through it. As such, the release hole 13 represents less than 1% of the total surface area of the casing composed of the base element 2 and the cover element 3.

During construction of the diagnostic device 1, the nitrocellulose membrane 10 is bonded to the testing portion 11 by way of a liquid-tight seal around the perimeter of the aperture 12. The seal around the perimeter of the aperture is liquid-tight during construction of the diagnostic device 1. That is to say, assembly of the diagnostic device 1 is not required in order for the seal to assume liquid-tight properties.

The liquid-tight seal around the perimeter of the aperture 12 is more clearly shown in FIG. 2 in which an alternative embodiment is depicted with like features shown with like numbers and hidden features shown in dashed lines. In this embodiment the diagnostic device 1 is depicted during construction. The nitrocellulose membrane element 10 is located on the cover element 3. Specifically, the nitrocellulose membrane element 10 is located between four corner brackets. The corner brackets 14 are located such that the aperture 12 is in the centre thereof. Thus, when the nitrocellulose membrane element 10 is located within the four corner brackets 14, the nitrocellulose membrane element 10 is aligned near perfectly so as to cover completely the aperture 12 and thereby allow for known consistency. This allows more accurate positioning of the nitrocellulose membrane element 10 such that the nitrocellulose membrane element 10 can be reduced in size, thus reducing the cost of the diagnostic device 1, whilst retaining the same construction tolerances.

In alternative embodiments, instead of the four corner brackets, a plurality (e.g. two or four) raised pegs are provided to facilitate positioning of the nitrocellulose membrane element 10.

At this point in construction, the nitrocellulose membrane element 10 is bonded to the testing portion 11 by way of a liquid-tight seal 15 around the perimeter of the aperture 12. It is to be appreciated that the liquid-tight seal 15 does not exactly follow the perimeter of the aperture 12 and is instead spaced slightly out from the perimeter of the aperture 12. In this way, a detection flow path is created from the outlet of the aperture 12 and into and through the nitrocellulose membrane element 10, whereas any bypass flow path from the outlet of the aperture 12 which bypasses the nitrocellulose membrane element 10 (in particular, a flow path going directly to the liquid-absorbent pad 9) is prevented.

In this embodiment, the liquid-tight seal 15 is formed by ultrasonic welding to form a molecular bond between the nitrocellulose membrane element 10 and the testing portion 11. However, in alternative embodiments, other means of forming a liquid-tight seal 15 may be used such as by use of an adhesive glue, an adhesive tape, liquid plastic or a 3D-printed bond. However, it is important that, whatever means of forming the liquid-tight seal 15 is employed, the seal completely surrounds the aperture 12 such that the only liquid flow path through the aperture 12 leads to the nitrocellulose membrane element 10.

In construction of the diagnostic device 1 shown in FIGS. 1 and 2, construction is completed by placing the base element 2 on the cover element 3 such that the respective upstanding peripheral lips 7,8 mutually engage (in embodiments in which a hinge 4 is provided, such placement involves folding the base element 2 and the cover element 3 about the hinge 4). The respective upstanding peripheral lips are then fixed to each other. In some embodiments, this is a pressure fit or friction fit. However, in preferred embodiments, the respective upstanding, peripheral lips 7,8 are ultrasonically welded together to form a liquid tight seal therebetween. In this way, the base element 2, the cover element 3 and the liquid-tight seal 15 define a chamber which is substantially liquid tight, the release hole 13 (which, as previously noted, forms less than 1% of the total surface area of the base element 2 and the cover element 3) permitting the transit of gases but not liquids (at standard pressure).

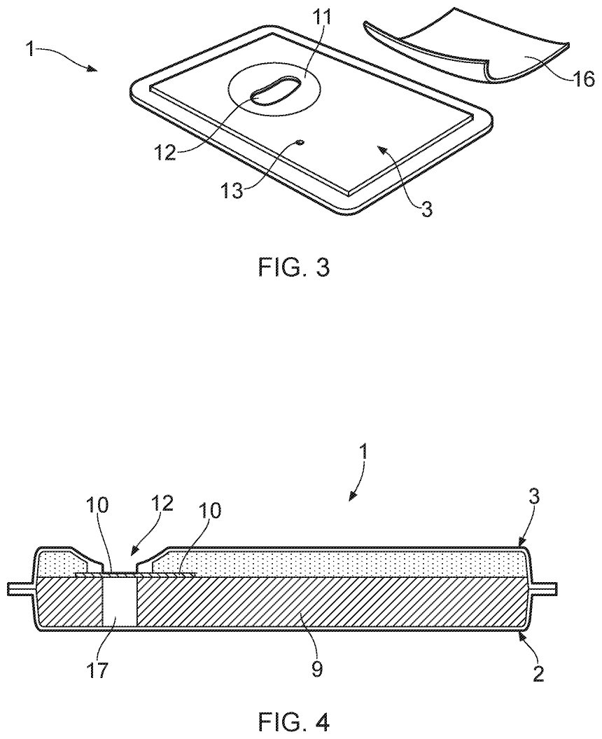

A final step in the construction of the diagnostic device 1 is shown in FIG. 3. This step applies to the embodiment shown in both FIGS. 1 and 2. A rectangular, flexible sealing sheet 16 is positioned over the testing section 11, specifically such that it covers the circular dimple thereof and the aperture 12. The sealing sheet 16 protects the nitrocellulose membrane element 10 prior to use.

In use of the constructed diagnostic device 1, a biological sample from an individual is obtained. Typically, the biological sample is a blood sample. However, in some embodiments, the biological sample is another antibody-containing sample such as sera or a saliva sample (saliva will typically contain IgA antibodies). The sealing sheet 16 is peeled off from the cover element 3 and the biological sample is deposited, via the aperture 12, on the nitrocellulose membrane element 10 which is thereby exposed.

In the case that the biological sample contains the antibody which is being detected, the antibody binds to the biological antigen which is immobilised on the nitrocellulose membrane element 10. Excess sample is absorbed through the nitrocellulose membrane element 10 and into the liquid-absorbent pad 9, firstly the blotting paper layer thereof and then into the cotton wadding material thereof. It is to be understood that, because of the presence of the liquid-tight seal 15, the only flow path for the biological sample is through the nitrocellulose membrane element 10; the liquid-tight seal 15 prevents escape of the biological sample via a flow path that bypasses the nitrocellulose membrane element 10 and leads directly to the liquid-absorbent pad 9.

In the case that the biological sample does not contain the antibody which is being detected, substantially all of the biological sample is absorbed through the nitrocellulose membrane element 10 and into the liquid-absorbent pad 9, without reaction with the biological antigen.

Subsequently, a detection reagent is deposited on the exposed section of the nitrocellulose membrane element 10, via the aperture 12. The detection reagent comprises a molecular conjugate which is composed of an anti-IgG or anti-IgM antibody conjugated to a gold particle. Thus, in the case that the biological sample contains the antibody, the molecular conjugate binds to the antibody which is, in turn, bound to the biological antigen immobilised on the nitrocellulose membrane element 10. The concentration of the gold particles from the molecular conjugate on the nitrocellulose membrane element 10 results in a detectable spot being visible on the nitrocellulose membrane element 10 within the aperture 12.

In the case that the biological sample does not contain the antibody which is being detected, the molecular conjugate in the detection reagent is absorbed through the nitrocellulose membrane element 10 and into the liquid-absorbent pad 9 and the gold particles are dispersed such that no spot becomes visible.

Thus the presence or absence of the spot is indicative of the presence or absence of antibodies specific for the biological antigen respectively and is thereby indicative of the individual from whom the biological sample has been obtained being exposed or not exposed to the biological antigen.

After the test has been completed, the sealing sheet 16 is reapplied to the cover element 3 of the casing so as to cover the aperture 12 prior to the diagnostic device 1 being discarded. In this way, the biological sample which has been deposited onto the diagnostic device 1 is held in a substantially sealed chamber so as to minimise any risk of cross-infection.

It has been observed that in the above embodiments, the presence of the liquid-tight seal 15 modifies the flow rate through the diagnostic device 1 such that around 5 to 10 minutes after the detection reagent is deposited on the nitrocellulose membrane element 10, the molecular conjugate and, specifically, the gold particles thereof, reintegrate with the nitrocellulose whether or not the antibody of interest is present in the biological sample. This can mean that the presence or absence of the spot is less easily determined.

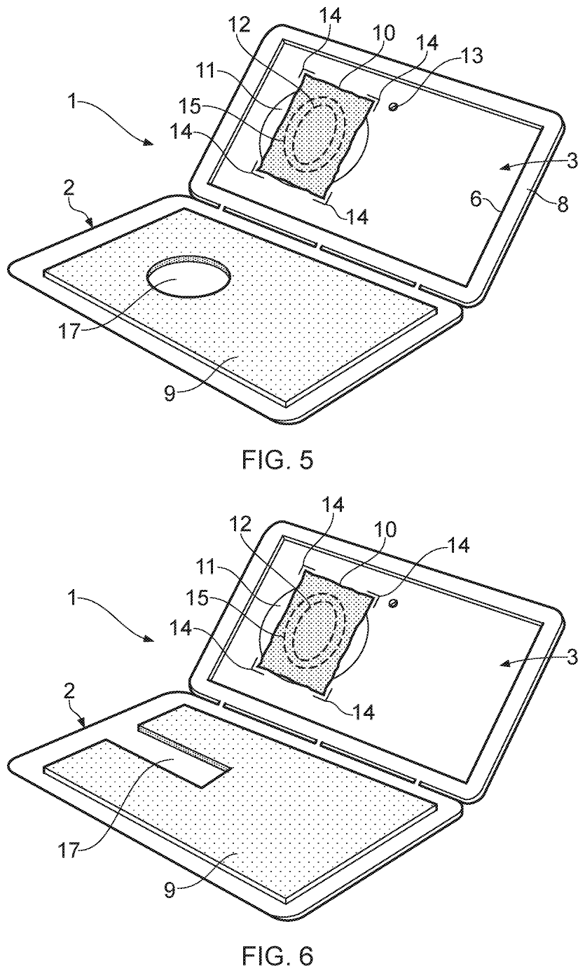

Accordingly, with reference to FIGS. 4 and 5, an alternative embodiment of the present invention will now be described in which like features are shown with like numbers. In this embodiment, the liquid-absorbent pad 9 has a circular opening 17 punched therein. The opening 17 is located such that, when the diagnostic device 1 is in its constructed form, the opening 17 is aligned with the aperture 12. Thus in the centre of the aperture 12, the nitrocellulose membrane element 10 is suspended above the opening 17. In this embodiment, the diagnostic device 1 is used substantially as described in relation to the previous embodiments. However, the presence of the opening 17 adjusts the flow rate of the biological sample through the diagnostic device 1. In particular, the presence of the opening 17 minimises or eliminates the reintegration of the molecular conjugate with the nitrocellulose membrane element 10, thus resulting in the presence or absence of the spot being more readily determined.

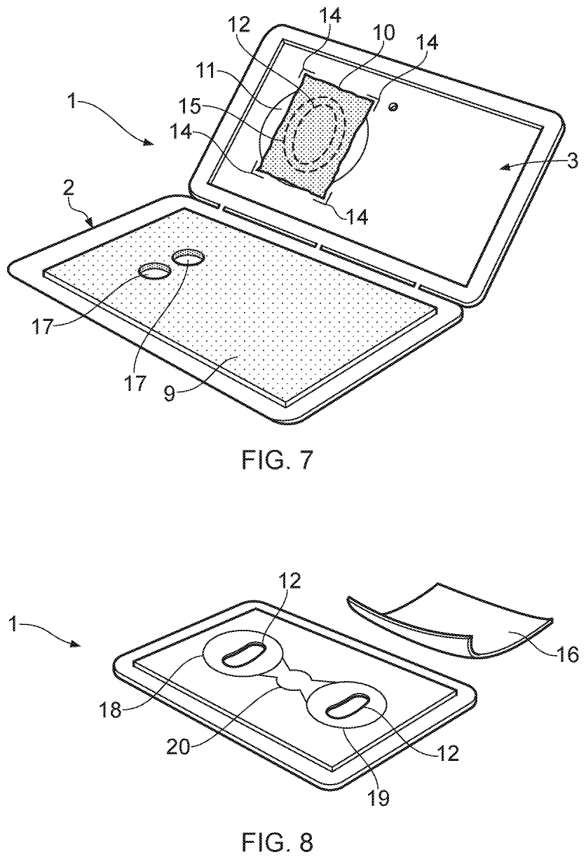

In the embodiment shown in FIGS. 4 and 5, the opening 17 is circular. However, in alternative versions of this embodiment, the opening 17 is a different shape. For example, as shown in FIG. 6, in one variant, the opening 17 is rectangular in shape and leads to a short side of the liquid-absorbent pad 9. In a further variant depicted in FIG. 7, two circular openings 17 are provided, aligned with the aperture 12.

In the above described embodiments, a single testing section 11 is provided on the diagnostic device 1. However, in alternative embodiments, a plurality of testing sections 11 are provided. For example, in the embodiment depicted in FIG. 8, two testing sections 18, 19 are provided, in liquid communication with a central well 20. Each of the testing sections 18, 19, has a respective aperture 12 and an associated nitrocellulose membrane (not shown) thereunder with a liquid-tight seal (not shown) around each aperture 12. Each of the respective nitrocellulose membranes has a different biological antigen immobilised thereon. In use, the sealing sheet 16 (which covers both of the testing sections 18, 19 and the central well 20) is removed and the liquid, biological sample is deposited on the central well 20. From the central well 20, equal volumes of the liquid, biological sample flow to each of the testing sections 18, 19 and the running of the diagnostic device test proceeds as described above. In one embodiment, the plurality of testing sections 11 are arranged in a grid on the diagnostic device 1. The grid arrangement enables a plurality of independent testing sections 11 to be provided on the diagnostic device 1. It is to be appreciated that such an embodiment permits the rapid testing for the presence of antibodies specific for each of the different biological antigens, in the liquid, biological sample, in a controlled manner.

In the above described embodiments, a liquid-tight seal 15 is provided around the perimeter of the aperture 12. However, it is to be understood that, in other embodiments, an alternative flow control structure is provided instead of a liquid-tight seal. For example, in some embodiments, the flow control structure is a gated periphery of raised dots of varying size and density. In such embodiments, the flow control structure does not prevent flow which bypasses the nitrocellulose membrane element 10 completely. Instead, it allows the passage of contaminating materials from the liquid, biological sample such as larger proteins and surfactants along a bypass flow path which leads directly to the liquid-absorbent pad 9, whereas the remainder of the biological sample passes along a detection flow path into the nitrocellulose membrane element 10. Such an arrangement prevents the contaminating materials from blocking the pores on the nitrocellulose membrane element. In further embodiments, the flow control structure comprises one or more levees or trenches around the periphery of the aperture 12 such that the seal is not completely liquid tight but, nevertheless, the liquid, biological sample is preferentially directed along the detection flow path into the nitrocellulose membrane 10 and there is a reduction in the flow of the sample that would, in the absence of the levees or trenches, pass along a bypass flow path directly to the liquid-absorbent pad 9. Such arrangements of raised dots, levees and/or trenches permit control and adjustment of the flow path of the biological sample.

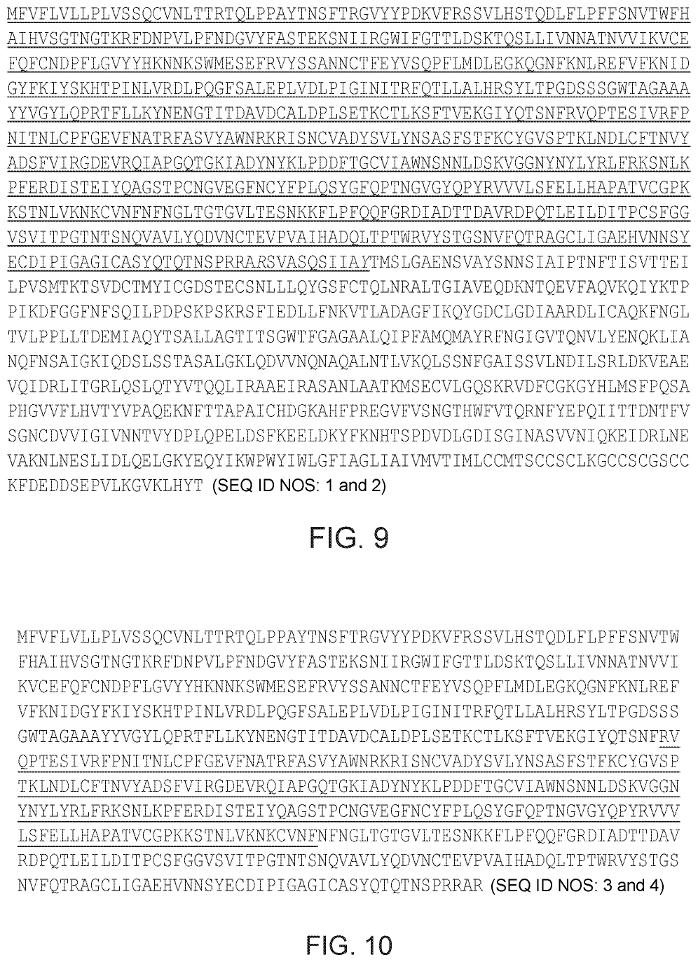

In the above embodiments, reference has been made to a biological antigen. It is preferred that the biological antigen is a COVID-19 protein or a fragment thereof. In some embodiments, the biological antigen comprises a spike protein, or fragment thereof, of COVID-19, the amino acid sequence of which is shown in FIG. 9 (SEQ ID NO: 2), preferably the S1 spike protein, or fragment thereof, depicted in FIG. 10 (SEQ ID NO: 4). The amino acid sequence of the spike protein of COVID-19 as depicted in FIG. 10 (SEQ ID NO: 4) corresponds to UniprotKB accession number P0DTC2 (SPIKE_SARS2). In some embodiments, a pool of different COVID-19 protein fragments (e.g. a pool of different S1 spike protein fragments) is provided as the biological antigen. In some embodiments, the biological antigen comprises additional sequences at the N-terminus or C-terminus such as a linker sequence to facilitate immobilisation on the nitrocellulose membrane element 10. In alternative embodiments, the biological antigen does not comprise the exact sequence from COVID-19 set out above but instead comprises a sequence with at least 70%, 80%, 90%, 95% or 99% sequence identity to the sequences set forth in FIG. 9 (SEQ ID NO: 2) or FIG. 10 (SEQ ID NO: 4). In embodiments of the present invention, the protein, or fragment thereof is at least 8, 10, 12, 14, 16, 18, 20, 30, 100, 200 or 300 amino acids long. It is to be understood that, by detecting the presence of antibodies specific for the COVID-19 protein or fragment thereof in the biological sample from an individual, it is indicative that the individual is currently, or has previously been, infected by the COVID-19 virus.

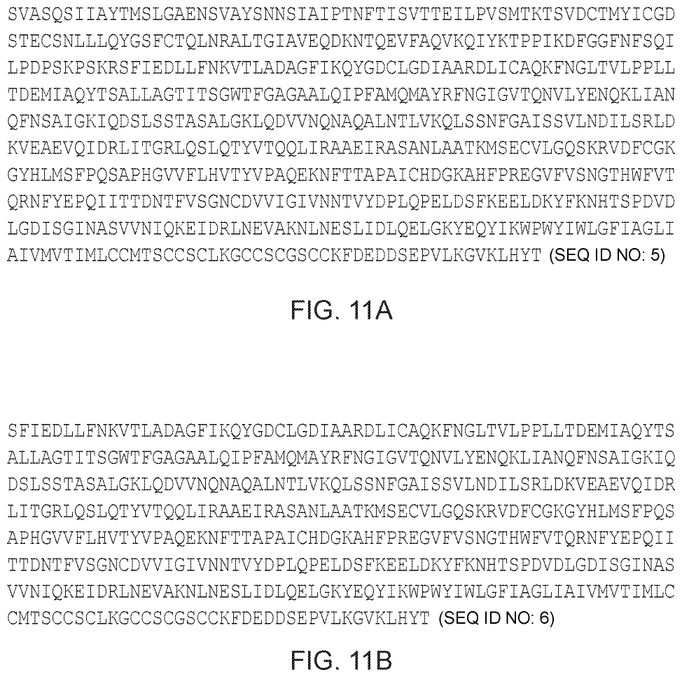

In one embodiment, the biological antigen comprises a spike protein, or a fragment thereof, of COVID-19 as described above. In a preferred embodiment, the biological antigen comprises the S1 spike protein, or a fragment thereof, depicted in FIG. 10 (SEQ ID NO: 4). In a further embodiment, the biological antigen comprises the S2 spike protein, or a fragment thereof. The sequence of the S2 spike protein is depicted in FIGS. 11A (SEQ ID NO: 5) and 11B (SEQ ID NO: 6). In one embodiment, the sequence of the S2 spike protein is edited such that the C-terminal 6 amino acids as depicted in either FIG. 11A (SEQ ID NO: 5) or 11B are (SEQ ID NO: 6) removed (i.e. “VKLHYT”). In one embodiment, the sequence of the S2 protein sequence is edited such that the C-terminal 62 amino acids as depicted in either FIG. 11A (SEQ ID NO: 5) or 11B (SEQ ID NO: 6) are removed. This edited version of the S2 spike protein is encompassed within the term “fragment thereof” of the S2 spike protein. In one embodiment, the S1 spike protein, or the fragment thereof, and/or the S2 spike protein, or the fragment thereof, is immobilised on the nitrocellulose membrane element 10 of the device 1. In one embodiment, the S1 and S2 spike proteins (that is to say, the sequence depicted in FIG. 10 [SEQ ID NO: 4] and the sequence depicted in FIG. 11A [SEQ ID NO: 5] or 11B [SEQ ID NO: 6]) or a fragment of either (such as the C-terminal edited version of the S2 spike protein) are comprised within a single polypeptide chain and the single polypeptide chain is immobilised on the nitrocellulose membrane element 10. In an alternative embodiment, the S1 and S2 spike proteins, or fragments of either, are immobilised at discrete sites on the nitrocellulose membrane element 10. That is to say, the S1 spike protein, or the fragment thereof, is immobilised at a first location on the nitrocellulose membrane element 10 and the S2 spike protein, or the fragment thereof, is immobilised at a second location on the nitrocellulose membrane element 10, wherein the first and the second locations are distinct and/or spatially separate from each other.

In one embodiment in which the biological antigen comprises the spike protein, or a fragment thereof, of COVID-19, the biological antigen is the trimeric spike protein. The trimeric spike protein of COVID-19 is a homotrimeric, class I fusion glycoprotein that mediates viral attachment, fusion, and entry into host cells. Each monomer contains an S1 and an S2 subunit (also referred to herein as the “S1 spike protein” and the “S2 spike protein” respectively) that mediate viral attachment and membrane fusion, respectively. In an alternative embodiment in which the biological antigen comprises the spike protein, or a fragment thereof, of COVID-19, the biological antigen is not the trimeric spike protein. That is to say, the spike protein immobilised on the nitrocellulose membrane element 10 is not a trimeric spike protein.

In a preferred embodiment, the spike protein, or the fragment thereof, comprises a COVID-19 S1 spike protein or a fragment thereof. In some embodiments, the COVID-19 S1 spike protein or the fragment thereof, is immobilised on the nitrocellulose membrane element 10 in isolation. In one embodiment, the COVID-19 S1 spike protein or the fragment thereof, is immobilised on the nitrocellulose membrane 10 in isolation from a further biological antigen. In one embodiment, “in isolation” refers to an absence of the further biological antigen being immobilised on the nitrocellulose membrane element 10 or to the separate positioning of the further biological antigen in relation to the COVID-19 S1 spike protein or the fragment thereof on the nitrocellulose membrane element 10. In one embodiment, the further biological antigen is a nucleoprotein, or a fragment thereof, of COVID-19 (as will be described below). In one embodiment, the further biological antigen is another component of the spike protein or a fragment thereof, such as the COVID-19 S2 spike protein or a fragment thereof.

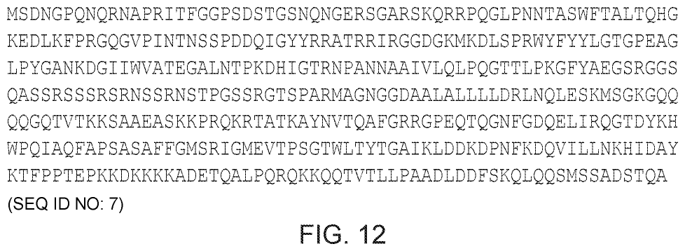

In one embodiment, a biological antigen is provided which comprises a nucleoprotein, or a fragment thereof, of COVID-19. In one embodiment, the nucleoprotein, or the fragment is further immobilised on the nitrocellulose membrane element 10. The sequence of the nucleoprotein is depicted in FIG. 12 (SEQ ID NO: 7) and corresponds to UniProt KB accession number P0DTC9 (NCAP_SARS2). In one embodiment, the nucleoprotein, or the fragment thereof, is immobilised at a discrete site from the S1 spike protein, or the fragment thereof, and/or the S2 spike protein, or the fragment thereof. In embodiments in which the nucleoprotein, or the fragment thereof, is immobilised on the nitrocellulose membrane element 10, its position on the nitrocellulose membrane element 10 and/or a label enables independent detection of an antibody specific for the spike protein, or the fragment thereof by the device 1. In some embodiments, it enables independent detection of an antibody specific for the S1 spike protein, or the fragment thereof and/or the S2 spike protein, or the fragment thereof.

In some embodiments, a pool of different COVID-19 protein fragments (e.g. a pool of different S1 spike protein fragments and/or different S2 spike fragments and/or different nucleoprotein fragments) is provided as the biological antigen. In some embodiments, the biological antigen comprises additional sequences at the N-terminus or C-terminus such as a linker sequence to facilitate immobilisation on the nitrocellulose membrane element 10. In one embodiment, the additional sequence is one or more of a His-tag, a His6-tag, an Fc-tag, a glycine-serine linker and/or a Strep-tag or Strep-tag II. In alternative embodiments, the biological antigen does not comprise the exact sequence from COVID-19 set out above but instead comprises a sequence with at least 70%, 80%, 90%, 95% or 99% sequence identity to a sequence set forth in any one of FIGS. 9 to 12 (SEQ ID NOS: 1 to 7). In one embodiment, the biological antigen consists of a sequence set forth in any one of FIGS. 9 to 12 (SEQ ID NOS: 1 to 7). In embodiments of the present invention, the protein, or fragment thereof is at least 8, 10, 12, 14, 16, 18, 20, 30, 100, 200 or 300 amino acids long.

It is to be understood that the term “COVID-19 protein or fragment thereof” encompasses the sequences of SEQ ID NOS: 1 to 7 (FIGS. 9 to 12), preferably any one or more of SEQ ID NOS: 1 to 6 (FIGS. 9 to 11A,B) and fragments thereof and sequences having at least 70%, 80%, 90%, 95% or 99% sequence identity thereto. Therefore, also encompassed are the sequences of SARS-CoV-2 variants (e.g. which emerge as the virus mutates during replication within infected individuals). Thus in one embodiment, the biological antigen comprises a sequence having at least 70% sequence identity and less than 100% sequence identity to the sequence of any one of SEQ ID NOS: 1 to 7 (also referred to herein as a “sequence variant”). Preferably, 80%, 85%, 90%, 95%, 99% sequence identity thereto. In one embodiment, the biological antigen comprises a sequence having one or more amino acid substitutions, deletions and/or insertions in the sequence of a COVID-19 protein or fragment thereof (also referred to herein collectively as a “mutation”). In one embodiment, the biological antigen comprises a sequence that is formed by substituting, deleting, and/or inserting 1 to 20, 1 to 15, 1 to 10, 1 to 8, 1 to 6, 1 to 5, 1 to 4, 1 to 3, 1 or 2, or 1 amino acid in the amino acid sequence of a COVID-19 protein or a fragment thereof.

In one embodiment, the biological antigen comprises a COVID-19 spike protein or a fragment thereof comprising one or more mutations as compared with the sequence of SEQ ID NO: 2 (FIG. 9). In one embodiment, the one more mutation is selected from: S13I, L18F, T20N, L21F, P26S, Q52R, a deletion at amino acid positions 69 and/or 70, D80A, 182T, L841, D138Y, a deletion at amino acid position 144, W152C, R190S, D215G, A222V, K417T, K417N, N439K, V445I, L452R, Y453F, S477N, E484K, E484Q, G485R, N501Y, A570D, E583D, D614G, P614R, H655Y, Q677H, P681H, A701V, T716I, F888L, S982A, T1027I, V1176F, D1118H and/or D1183Y.

In a preferred embodiment, the biological antigen comprises a COVID-19 S1 receptor binding domain comprising one or more mutations as compared with the sequence of SEQ ID NO: 3 (underlined in FIG. 10). In one embodiment, the one more mutation is selected from: K417N, K417T, N439K, V445I, L452R, Y453F, S477N, E484K, E484Q, G485R, or N501Y. More preferably K417N, K417T, E484K, E484Q and/or N501Y.

In one embodiment, the biological antigen comprises a COVID-19 S1 spike protein or a fragment thereof comprising a sequence in which one or more amino acids are mutated in a sequence consisting of the N-terminal 100, 200, 300, 400, 500 or 600 amino acids of the sequence of SEQ ID NO: 4 (as represented in FIG. 10). In a preferred embodiment, the mutation is a substitution. In a further embodiment, the sequence further contains one or more deletions and/or insertions.

In one embodiment, the biological antigen comprises 1, 2, 3, 4, 5, 6, 7, 8, 10, 15 or 20 of the mutations set out above.

In one embodiment, the COVID-19 S1 spike protein or the fragment thereof is a polypeptide comprising the sequence of SEQ ID NO: 4 (as shown in FIG. 10) or a sequence comprising amino acids 13 to 685, 1 to 674, 13 to 674, 1 to 682, or 13 to 682 of the sequence of SEQ ID NO: 4. In one embodiment, the sequence consists of amino acids 13 to 685, 1 to 674, 13 to 674, 1 to 682, or 13 to 682 of the sequence of SEQ ID NO: 4. In one embodiment, the COVID-19 S1 spike protein or the fragment thereof comprising or consisting of the sequence of SEQ ID NO: 4, or comprising or consisting of amino acids 13 to 685, 1 to 674, 13 to 674, 1 to 682, or 13 to 682 of the sequence of SEQ ID NO: 4, comprises one or more of the mutations as set out above.

In one embodiment, the COVID-19 spike protein or the fragment thereof is a polypeptide comprising the sequence of SEQ ID NO: 2 (as shown in FIG. 9) or a sequence comprising amino acids 13 to 1273, 1 to 890, 13 to 890, 1 to 985, 13 to 985, 1 to 1030, 13 to 1030, 1 to 1120, 13 to 1120, 1 to 1180, 13 to 1180, 1 to 1205, 13 to 1205, 1 to 1211, or 13 to 1211 of the sequence of SEQ ID NO: 2. In one embodiment, the sequence consists of amino acids 13 to 1273, 1 to 890, 13 to 890, 1 to 985, 13 to 985, 1 to 1030, 13 to 1030, 1 to 1120, 13 to 1120, 1 to 1180, 13 to 1180, 1 to 1205, 13 to 1205, 1 to 1211, or 13 to 1211 of the sequence of SEQ ID NO: 2. In one embodiment, the COVID-19 spike protein or the fragment thereof comprising or consisting of the sequence of SEQ ID NO: 2, or comprising or consisting of amino acids 13 to 1273, 1 to 890, 13 to 890, 1 to 985, 13 to 985, 1 to 1030, 13 to 1030, 1 to 1120, 13 to 1120, 1 to 1180, 13 to 1180, 1 to 1205, 13 to 1205, 1 to 1211, or 13 to 1211 of the sequence of SEQ ID NO: 2, comprises one or more of the mutations as set out above.

In one embodiment, the COVID-19 spike protein or the fragment thereof comprises one or more mutations in the amino acid sequence corresponding to the cleavage site between the S1 and S2 subunits. In one embodiment, the one or more mutations are in the furin cleavage site corresponding to amino acids 682 to 685 of the amino acid sequence of SEQ ID NO: 2. In one embodiment, the one or more mutations reduce or prevent cleavage of the S1 and S2 subunits into separate polypeptides. In one embodiment, the COVID-19 spike protein or the fragment thereof comprises one or more stabilising mutations. In one embodiment, the one or more stabilising mutations comprise a proline substitution. In one embodiment, the one or more stabilising mutations comprise the HexaPro mutations as described in Hsieh et al., Science 369, 1501-1505 (2020). In one embodiment, the COVID-19 spike protein or the fragment thereof contains 6 amino acid changes to proline and a C-terminal T4 fibritin trimerization domain to stabilize the protein in its pre-fusion conformation as described in Hsieh et al. (ibid).

In the embodiments described above, the COVID-19 protein or the fragment thereof that is immobilised on the nitrocellulose membrane element 10 is produced in insect cells. In an alternative embodiment, the COVID-19 protein or the fragment thereof that is immobilised on the nitrocellulose membrane element 10 is produced in mammalian cells, preferably sheep cells. In a further embodiment, an alternative host cell is used for protein expression of the COVID-19 protein or the fragment thereof.

In certain embodiments, a control is immobilised on the nitrocellulose membrane element 10. In a preferred embodiment, the control comprises a positive control. In one embodiment, the positive control is an anti-IgG antibody, preferably an anti-human IgG antibody. In an alternative embodiment, a control other than an anti-IgG antibody is immobilised on the nitrocellulose membrane element 10.

In use of the diagnostic device 1, a biological sample from an individual is obtained. Typically, the biological sample is a blood sample. However, in some embodiments, the biological sample is another antibody-containing sample such as sera or a saliva sample. The biological sample is deposited, via the aperture 12, on the nitrocellulose membrane element 10.

In the case that the biological sample contains the antibody which is being detected, the antibody binds to the biological antigen which is immobilised on the nitrocellulose membrane element 10. Excess sample is absorbed through the nitrocellulose membrane element 10. In the case that the biological sample does not contain the antibody which is being detected, substantially all of the biological sample is absorbed through the nitrocellulose membrane element 10 without reaction with the biological antigen.

In embodiments in which a control comprising an anti-IgG antibody, is immobilised on the nitrocellulose membrane element 10, any IgG antibody (i.e. regardless of the specific target of the IgG antibody) contained in the biological sample will bind to the anti-IgG antibody of the control.

Subsequently, a detection reagent is deposited on the exposed section of the nitrocellulose membrane element 10, via the aperture 12. The detection reagent comprises a molecular conjugate which is composed of an anti-IgG antibody conjugated to a gold particle. In an alternative embodiment, the molecular conjugate is composed of an anti-IgM antibody conjugated to a gold particle (in such an embodiment, the control comprises an anti-IgM antibody). Thus, in the case that the biological sample contains the antibody, the molecular conjugate binds to the antibody which is, in turn, bound to the biological antigen immobilised on the nitrocellulose membrane element 10. The concentration of the gold particles from the molecular conjugate on the nitrocellulose membrane element 10 results in a detectable spot being visible on the nitrocellulose membrane element 10 within the aperture 12. Similarly, the molecular conjugate binds to the IgG antibody bound to the anti-IgG antibody of the control, resulting in a detectable area corresponding to the control being visible on the nitrocellulose membrane element 10. This acts as a positive control to indicate that the diagnostic test is functioning appropriately.

In the case that the biological sample does not contain the antibody which is being detected, the molecular conjugate in the detection reagent is absorbed through the nitrocellulose membrane element 10 and the gold particles are dispersed such that no spot becomes visible.

Thus the presence or absence of the spot is indicative of the presence or absence of antibodies specific for the biological antigen respectively and is thereby indicative of the individual from whom the biological sample has been obtained being exposed or not exposed to the biological antigen.

In embodiments in which the nitrocellulose membrane element 10 comprises the S1 spike protein, or the fragment thereof, and/or the S2 spike protein, or the fragment thereof immobilised thereon at discrete sites, the presence of a spot at one or each of these discrete sites is indicative of the presence of antibodies specific for the S1 spike protein, or the fragment thereof, and/or the S2 spike protein, or the fragment thereof. This is thereby indicative of the individual from whom the biological sample has been obtained being currently or previously infected by COVID-19 or having been vaccinated against COVID-19.

In embodiments in which the nitrocellulose membrane element 10 further comprises the nucleoprotein, or the fragment thereof, immobilised thereon at a further discrete site, the presence of a spot at this discrete site combined with the absence of a spot at each of the sites corresponding to the S1 spike protein, or the fragment thereof, and the S2 spike protein, or the fragment thereof, is indicative of the presence of antibodies specific for the nucleoprotein, or the fragment thereof only. That is to say, it is indicative of the absence of antibodies specific for the S1 spike protein, or the fragment thereof, and the S2 spike protein, or the fragment thereof. The sequence of the spike protein is more specific to COVID-19, whereas the sequence of the nucleoprotein is more conserved between the beta group of coronaviruses. Therefore, the presence of antibodies specific for the nucleoprotein, or the fragment thereof, combined with the absence of antibodies specific for a spike protein, or a fragment thereof, is indicative of the individual from whom the biological sample has been obtained being currently or previously infected by a beta group coronavirus other than COVID-19 or having been vaccinated against a beta group coronavirus other than COVID-19.

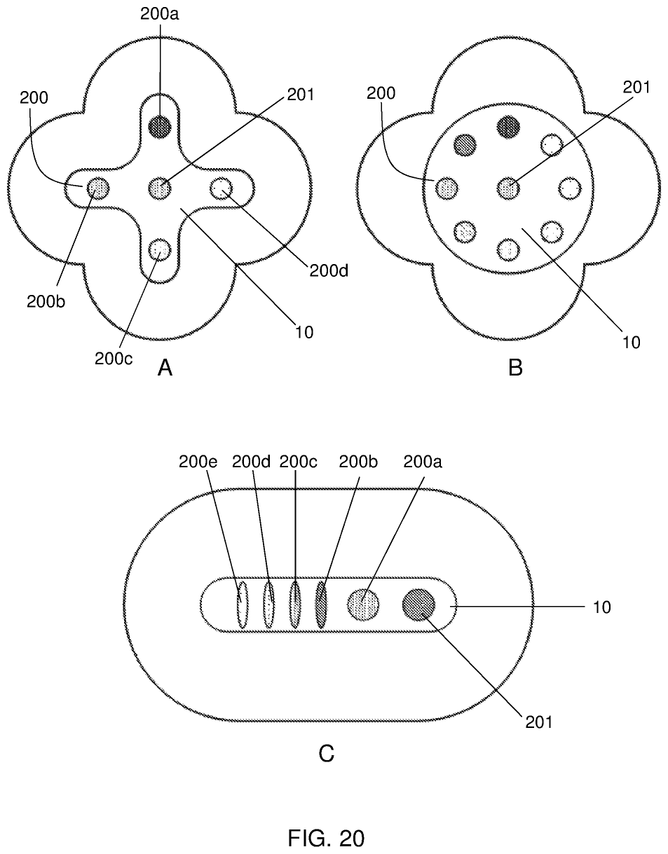

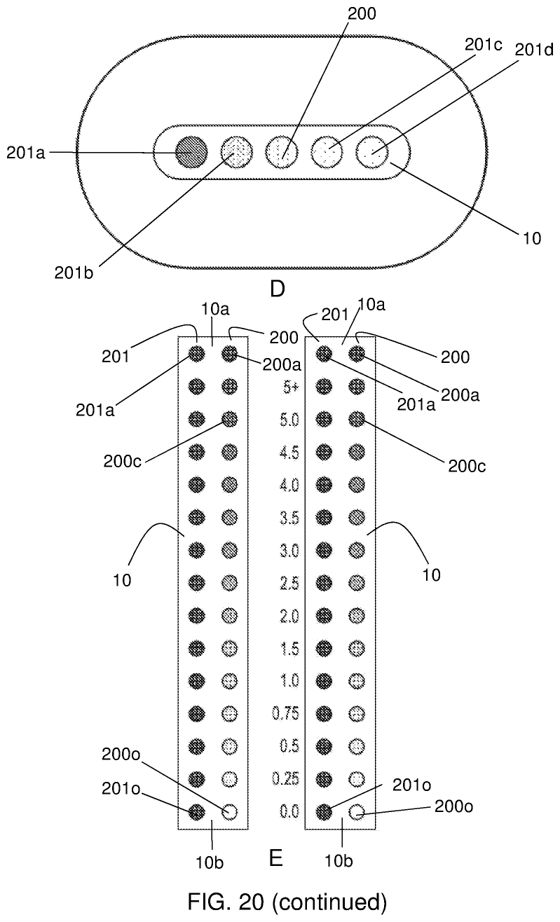

Referring to FIG. 20A, a further embodiment of the present invention will now be described. The nitrocellulose membrane element 10 comprises a detection portion 201 on which a biological antigen is immobilised and a plurality of reference elements 200. In this embodiment, a first, a second, a third and a fourth reference element 200a, 200b, 200c, 200d are provided on the nitrocellulose membrane element 10, each at a discrete site and at a position such that they surround the detection portion 201. Each reference element 200a-d comprises a quantity of IgG antibody immobilised thereon. An increased amount of IgG antibody is immobilised on the first reference element 200a and sequentially less IgG antibody is immobilised on the second to fourth reference elements 200b-c. In use, after a liquid, biological sample is applied to the nitrocellulose membrane element 10, a detection reagent is also applied. If the liquid, biological sample contains antibodies specific to the biological antigen, which is immobilised on the nitrocellulose membrane element 10, then these antibodies bind to the biological antigen and are also immobilised on the nitrocellulose membrane element 10. In this embodiment, the detection reagent comprises a gold particle conjugated to an anti-IgG antibody. The anti-IgG antibody in the detection reagent binds to the antibody specific to the biological antigen immobilised at the detection portion 201 and also to the IgG antibody immobilised at each of the reference elements 200a-d, thus concentrating the gold particles onto the nitrocellulose membrane element 10 such that a detectable spot is visible at the detection portion 201 and the reference elements 200a-d. The intensity of the detectable spot that is visible at the detection portion 201 is compared visually with the intensity of the detectable spot at each of the first to fourth reference elements 200a-d. In FIG. 20A, the intensity of the detectable spot at the detection portion 201 is substantially equivalent to that at the second reference element 200b. This indicates that the level of antibody present in the liquid, biological sample represents a moderate positive result. If the intensity of the detectable spot at the detection portion 201 were to be substantially equivalent to one of the first, third or fourth reference elements instead 200a, c or d, this would indicate that the level of antibody present in the liquid, biological sample represented a strong positive result, a weak positive result or a equivocal (uncertain if positive or negative) result respectively. Thus a visual comparison of the intensity of the detectable spot at the detection portion 201 with the reference elements 200a-d provides a semi-quantitative indication of the level of antibody in the liquid, biological sample.

Referring to FIG. 20B, a variant of the above embodiment is shown in which like features are shown with like numbers. In this variant embodiment, an increased number of reference elements 200 are provided on the nitrocellulose membrane element 10. The reference elements are positioned on the nitrocellulose membrane element 10 at a series of discrete sites, in a circular pattern with a detection portion 201 positioned substantially at the centre thereof. In use, this variant embodiment operates in substantially the same manner as described in relation to FIG. 20A, except that an increased number of visual comparison points are provided by the increased number of reference elements 200. This provides further detail on the level of antibody in the liquid, biological sample.

Referring to FIGS. 20C and 20D, further variant embodiments are shown in which like features are shown with like numbers. Referring to FIG. 20C, a detection portion 201 is provided on a nitrocellulose membrane element 10 together with a plurality of reference elements 200a-e. The reference elements 200a-e comprise IgG immobilised over a substantially circular area 200a or in a series of discrete lines 200b-e. In use, this arrangement produces a detectable spot at reference element 200a or a series of detectable lines at reference elements b-e, following application of the detection reagent. The intensity of the detectable spot that is visible at the detection portion 201 can be compared visually with the intensity of the detectable spot or lines that are visible at the reference elements 200a-e to provide an indication of the level of antibody present in the liquid, biological sample. In alternative embodiments, only the reference element 200a, comprising IgG immobilised over a substantially circular area, or the reference elements 200b-e, comprising IgG immobilised in a series of discrete lines, are provided on the nitrocellulose membrane element 10 with the detection portion 201.

Referring to FIG. 20D, in this embodiment, a single reference element 200 is provided on the nitrocellulose membrane element 10, together with a plurality of detection portions 201a-d. In one embodiment, a different biological antigen is immobilised on each detection portion 201a-d. In use, a liquid biological sample is applied to the nitrocellulose membrane element 10 and any antibodies present in the sample that are specific for a biological antigen immobilised on a detection portion 201a-d will bind thereto. If such antibodies are present then following application of the detection reagent, the intensity of a detectable spot at a detection portion 201a-d is compared with the intensity of the detectable spot at the reference element 200 to provide an indication of the level of antibody in the liquid biological sample specific for the antigen immobilised at each detection portion 201a-d.

Referring to FIG. 20E, a nitrocellulose membrane element 10 comprising a first and a second end portion 10a, 10b is provided with a plurality of detection portions 201, each comprising a biological antigen immobilised thereon, and a plurality of reference elements 200. The plurality of detection portions 201 and the plurality of reference elements 200 are provided at a series of discrete sites which extend between the first and second end portions 10a, 10b of the nitrocellulose membrane element 10, with each detection portion 201a adjacent to a reference element 200a on the nitrocellulose membrane element 10. Each reference element 200 comprises a quantity of IgG antibody immobilised thereon. In this embodiment, a first reference element 200a, located towards the first end portion 10a of the nitrocellulose membrane element 10, comprises the highest concentration of IgG antibody immobilised thereon and a second reference element 200o, located towards the second end portion 10b of the nitrocellulose membrane element 10, comprises the lowest concentration of IgG antibody immobilised thereon, with the series of discrete sites therebetween having sequential concentrations thereon. FIG. 20E illustrates that a series of reference elements 200a-o are able to provide a semi-quantitative scale to indicate the level of antibody in a liquid, biological sample. In this instance, the intensity of the detectable spot that is visible at each of the detection portions 201 is substantially equivalent to the intensity of the detectable spot of the reference element 200c corresponding to a “level 5”. This indicates a strong positive result, suggesting that the level of antibody in the liquid, biological sample is high. Conversely, if the intensity of the detectable spot visible at the detection portions 201 were substantially equivalent to the intensity of a detectable spot of a reference element 200 corresponding to a lower level then this would indicate a moderate or weak positive result or an equivocal result, suggesting that the level of antibody in the liquid, biological sample was lower.

It is to be understood that in some embodiments, a single reference element 200 is provided and a visual comparison of the intensity of a detectable spot at a detection portion 201 is used to indicate whether or not the intensity is higher, equivalent or lower than that of the reference element 200. This is then used to infer the level of antibody in the liquid, biological sample. In other embodiments, a plurality of reference elements 200 are provided and a visual comparison of the intensity of a detectable spot at a detection portion 201 is made across the plurality of reference elements 200. The intensity of the detectable spot at the detection portion 201 which most closely matches that at a reference element 200 is then used to infer the level of antibody in the liquid, biological sample. It is to be appreciated that an advantage of including the reference element 200 on the same nitrocellulose membrane element 10 as the detection portion 201 is that any variables (e.g. temperature, humidity, flow rate) that might affect the intensity of the detectable spot or line that forms at the detection portion 201 will also affect the intensity of the detectable spot or line that forms at the reference element 200 in a substantially proportional manner. Thus the presence of the reference element 200 on the same nitrocellulose membrane element 10 as the detection portion 201 provides a control for certain variables which might otherwise affect the accuracy of the semi-quantitative indication of the level of antibody in the liquid, biological sample.

In the above embodiments and variants thereof the reference element 200 has been described as comprising an IgG antibody, which is immobilised across a substantially circular area or in a line. In an alternative embodiment, the reference element 200 comprises an alternative moiety and the detection reagent comprises a molecular conjugate comprising an alternative binding moiety that is complementary thereto. In one embodiment, the reference element comprises an alternative antibody (e.g. IgM) and the molecular conjugate comprises an alternative complementary antibody (e.g. anti-IgM). In further alternative embodiments, the IgG antibody or alternative moiety comprised within the reference element 200 is immobilised across an area that is an alternative shape (e.g. rectangular, square, oval etcetera).

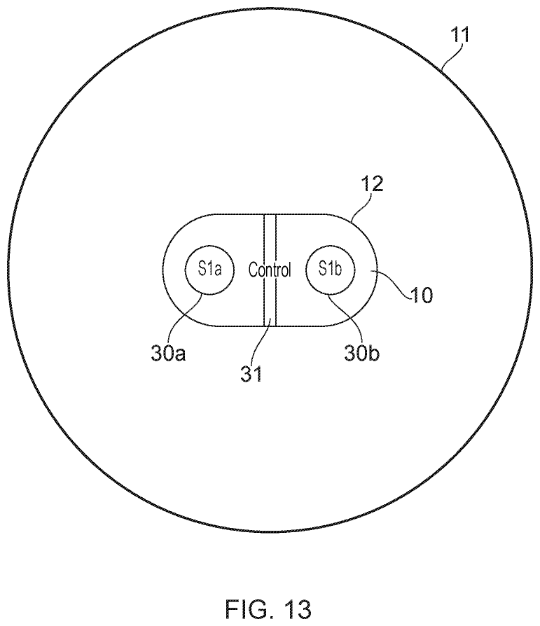

Referring to FIG. 13, a further embodiment of the present invention will now be described in which like features are shown with like numbers. A testing portion 11 comprises a circular dimple such that the dimple is convex towards a nitrocellulose membrane element 10. In the centre of the circular dimple is a lozenge-shaped aperture 12. In alternative embodiments, the aperture 12 is an alternative shape such as being rectangular, circular, square et cetera.

The nitrocellulose membrane element 10 comprises a first site 30a and a second site 30b at which a biological antigen and a variant of the biological antigen are immobilised respectively. The first site 30a is discrete (that is to say, spatially separated) from the second site 30b on the nitrocellulose membrane element 10. In one embodiment, the area over which the biological antigen and the variant of the biological antigen is immobilised at the first site 30a and the second site 30b respectively is substantially circular in shape. In alternative embodiments, the area over which the biological antigen and/or the variant is immobilised is an alternative shape. In a further embodiment, a plurality of variants are immobilised on the nitrocellulose membrane element 10 (not shown), each at a discrete site. In a preferred embodiment, each variant is a different variant of the biological antigen immobilised at the first site 30a.

In one embodiment, the biological antigen and the variant of the biological antigen are the S1 spike protein, or the fragment thereof, of COVID-19 as described above, which have been produced in different host cells. That is to say, the biological antigen and the variant of the biological antigen have been produced in first and second host cells respectively, wherein the first and the second host cell are each from a different species. In a preferred embodiment, the biological antigen is the S1 spike protein, or the fragment thereof, which has been produced in an insect cell and the variant of the biological antigen is the S1 spike protein, or the fragment thereof, which has been produced in a mammalian cell. In an alternative embodiment, the biological antigen, which is the S1 spike protein, or the fragment thereof, is produced in the mammalian cell and the variant of the biological antigen is produced in the insect cell. In one embodiment, the mammalian cell is a sheep cell. In a further alternative embodiment, the biological antigen and/or the variant of the biological antigen is produced in an alternative host cell (e.g. other than a mammalian or insect cells). In one embodiment, the fragment of the S1 spike protein is the receptor binding domain of the S1 spike protein.

In one embodiment, the production of the biological antigen and the variant in a different host cell removes a contaminant associated with one of the biological antigen or the variant. In one embodiment, the production of the variant in a different host cell from the biological antigen results in the variant being a sequence variant and/or comprising an alternative post-translational modification compared with the biological antigen. In one embodiment, the variant is a sequence variant and comprises a sequence having at least 70% sequence identity and less than 100% sequence identity to the sequence of the biological antigen. Preferably, 80%, 85%, 90%, 95%, 99% sequence identity. In a further embodiment, the variant comprises an alternative post-translational modification compared with the biological antigen. That is to say, a modification of a protein or polypeptide that occurs subsequent to biosynthesis. In one embodiment, a post-translational modification is present on the variant but absent on the biological antigen or vice versa. Post-translational modifications include various chemical and/or biological (e.g. enzymatic) modifications, such as glycosylation.

In one embodiment, the biological antigen and the variant are produced in the same host cell and the variant is nevertheless a sequence variant. In one embodiment, the variant comprises a sequence having one or more amino acid substitutions, deletions and/or insertions in the sequence of a COVID-19 protein or fragment thereof (also referred to herein collectively as a “mutation”). In one embodiment, the COVID-19 protein or fragment thereof comprises any one of the sequence of SEQ ID NOS: 1 to 7 (FIG. 9 to 12) or a fragment thereof. In one embodiment, the variant comprises a sequence having at least 70% sequence identity and less than 100% sequence identity to the sequence of any one of SEQ ID NOS: 1 to 7 or a fragment thereof. In one embodiment, the biological antigen comprises: (a) a COVID-19 S1 spike protein or a fragment thereof, preferably wherein the COVID-19 S1 spike protein or the fragment thereof is a polypeptide comprising a sequence of at least 8, 10, 12, 14, 16, 18, 20, 30, 100, 200 or 300 amino acids from an amino acid sequence having at least 70%, 80%, 90%, 95%, 99% or 100% sequence identity to the sequence of SEQ ID NO: 4 (as represented in FIG. 10); and the variant comprises: (b) a biological antigen comprising a sequence having at least 70% sequence identity and less than 100% sequence identity to (a). It is to be understood that the term “variant” encompasses the sequences of SARS-CoV-2 variants as described above.

In one embodiment, a control is immobilised at a third site 31 on the nitrocellulose membrane element 10. In one embodiment, the control is immobilised over an area that defines a line between the first and second sites 30a, 30b. In an alternative embodiment, the control is immobilised over an area that is an alternative shape and/or position on the nitrocellulose membrane element 10. In a preferred embodiment, the control comprises a positive control. In one embodiment, the positive control is an anti-IgG antibody, preferably an anti-human IgG antibody. In an alternative embodiment, a control other than an anti-IgG antibody is immobilised on the nitrocellulose membrane element 10.

In a further embodiment, the biological antigen and the variant of the biological antigen as described above are a first biological antigen and a first variant. In this embodiment, a second biological antigen and a second variant of the biological antigen are further located at a fourth and fifth site respectively on the nitrocellulose membrane element 10 (not shown). The fourth site is discrete from the fifth site on the nitrocellulose membrane element 10. In this embodiment, the second biological antigen and the second variant are the S2 spike protein, or the fragment thereof, of COVID-19 as described above, which have been produced in different host cells. In yet a further embodiment, a third biological antigen and a third variant of the biological antigen are further located at a sixth and seventh site respectively on the nitrocellulose membrane element 10 (not shown). The sixth site is discrete from the seventh site on the nitrocellulose membrane element 10. In this embodiment, the third biological antigen and the third variant are the nucleoprotein, or the fragment thereof, of COVID-19 as described above, which have been produced in different host cells. In one embodiment, it is to be understood that each of the first and second sites 30a, 30b as well as the third to seventh sites are each discrete from each other.

In the further embodiments described above, the second and/or third biological antigens, which are the S2 spike protein, or the fragment thereof, and the nucleoprotein, or the fragment thereof respectively, have been produced in an insect cell and the variants of these biological antigens have been produced in a mammalian cell. In an alternative embodiment, the second and/or third biological antigens, which are the S2 spike protein, or the fragment thereof, and the nucleoprotein, or the fragment thereof, respectively, are produced in the mammalian cell and the variants of these biological antigens are produced in the insect cell. In one embodiment, the mammalian cell is a sheep cell. In a further alternative embodiment, the second and/or third biological antigen and/or the variant of the second and/or third biological antigen is produced in an alternative host cell (e.g. other than a mammalian or insect cells).

In a further embodiment, four, five, six or more biological antigens and variants thereof are immobilised on the nitrocellulose membrane element 10. In one embodiment, each of the biological antigens and variants as described above is immobilised at a discrete (preferably, spatially separate) site on the nitrocellulose membrane element 10. It is to be understood that by immobilising the biological antigens and their respective variants at discrete (preferably, spatially separate) sites on the nitrocellulose membrane element 10, an antibody specific for each is separately detectable. In an alternative embodiment, the biological antigens and their respective variants as described above are not immobilised at discrete sites and an alternative means enables an antibody specific for each to be separately detectable (e.g. a label). In one embodiment, the area over which the biological antigens and their respective variants is immobilised (e.g. at the first, second 30a, 30b and fourth to seventh sites) is substantially circular in shape. In alternative embodiments, the area over which the biological antigens and/or their respective variants is immobilised is an alternative shape.

In use of the diagnostic device of FIG. 13, in which the nitrocellulose membrane 10 comprises the biological antigen and the variant of the biological antigen immobilised thereon at discrete sites, the presence of a spot at each of these discrete sites is indicative of the presence of antibodies specific for the biological antigen and the variant thereof. Without wishing to be bound by theory, it is thought that certain individuals may possess antibodies specific for the biological antigen when produced in a certain host cell (e.g. as a result of a contaminant). This can produce a false positive on a diagnostic test. However, it is less likely that the individual would also possess antibodies specific for the variant of the biological antigen, which is produced in an alternative host cell. By requiring the presence of a spot at both of the sites corresponding to the biological antigen and variant in order to determine that antibodies specific for the biological antigen are, in fact, present in the sample, the incidence of false positives is reduced and thus the specificity of the diagnostic test is improved overall. Therefore, the presence of a spot at both of the sites corresponding to the biological antigen and variant is indicative of the presence of antibodies that are, in fact, specific for the biological antigen and is thereby indicative of the individual from whom the biological sample has been obtained being exposed to the biological antigen. The absence of a spot at both of the sites corresponding to the biological antigen and variant is indicative of the absence of antibodies that are, in fact, specific for the biological antigen and is thereby indicative of the individual from whom the biological sample has been obtained not having been exposed to the biological antigen. The presence of a spot at one of the sites and the absence of a spot at the other site is indicative of the probable absence of antibodies that are, in fact, specific for the biological antigen and is thereby indicative that the individual from whom the biological sample has been obtained has probably not been exposed to the biological antigen but may require further testing. On binding to IgG antibodies contained in the biological sample, the control 31, which defines a line between the spots at which the biological antigen and the variant are immobilised, becomes visible as described above. This positioning of the control 31 facilitates interpretation of the result from each spot.

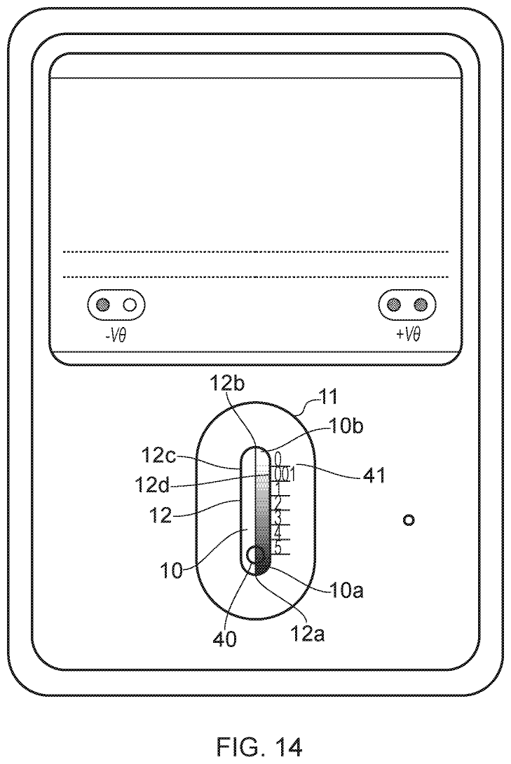

Referring to FIG. 14, a further embodiment of the present invention will now be described in which like features are shown with like numbers. A testing portion 11 comprises a lozenge-shaped area, centred within which is a lozenge-shaped aperture 12 defined by a first and a second end portion 12a, 12b and a first and a second side portion 12c, 12d. The testing portion 11 is convex towards a nitrocellulose membrane element 10. A series of graduation marks 41 are aligned with and extend alongside the second side portion 12d that defines the aperture 12. In alternative embodiments, the area of the testing portion 11 and/or the aperture 12 is an alternative shape such as being rectangular, circular, square etcetera. (The “−Vθ” and “+Vθ” control spots may be omitted from the device shown in FIG. 14 in some embodiments.)

The nitrocellulose membrane element 10 comprises a biological antigen immobilised thereon at a plurality of different concentrations. In one embodiment, the biological antigen comprises a spike protein, or fragment thereof, of COVID-19 as depicted in FIG. 9 (SEQ ID NO: 1 or 2). In an alternative embodiment, the biological antigen comprises any one of the biological antigens, or the fragments thereof, of COVID-19 as described above, such as the S1 spike protein, or a fragment thereof, as depicted in FIG. 10 (SEQ ID NO: 3 or 4) and/or the S2 spike protein, or a fragment thereof, as depicted in FIG. 11A (SEQ ID NO: 5) or FIG. 11B (SEQ ID NO: 5). The plurality of different concentrations of the biological antigen extend from a first point 10a on the nitrocellulose membrane element 10, located towards the first end portion 12a of the aperture 12, at which the concentration of the biological antigen is highest, through to a second point 10b on the nitrocellulose membrane element 10, located towards the second end portion 12b of the aperture 12, at which the concentration of the biological antigen is lowest, and at sequential concentrations therebetween.

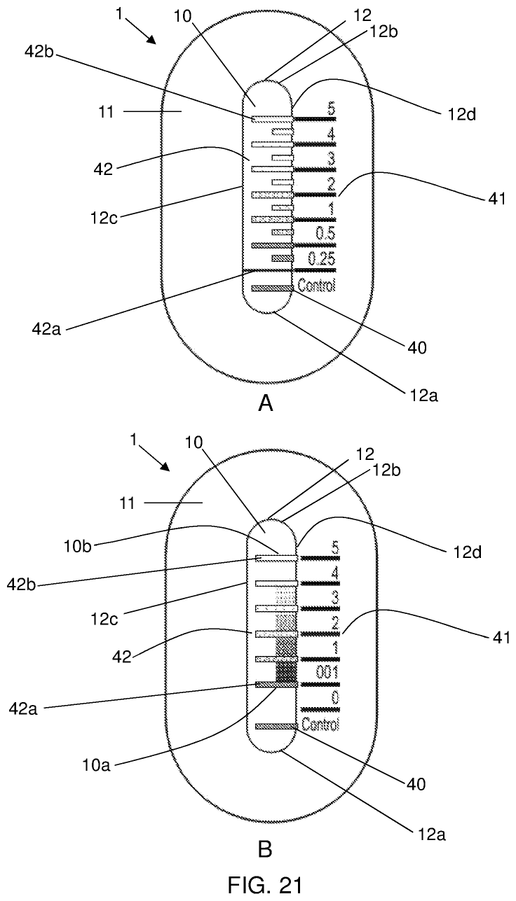

In one embodiment, the plurality of different concentrations of the biological antigen extend between the first and second points 10a, 10b on the nitrocellulose membrane element 10 in a substantially continuous gradation of their respective concentrations. By way of example, such an embodiment is constructed by spraying the biological antigen onto the nitrocellulose membrane element 10 between the first and second points 10a, 10b, such that the greatest amount of antigen is immobilised at the first point 10a and sequentially less antigen is immobilised as the second point 10b is approached. In an alternative embodiment, the plurality of different concentrations of the biological antigen extend between the first and second points 10a, 10b on the nitrocellulose membrane element 10 at a series of discrete sites (not shown). FIGS. 21A and 21B provide further detail on examples of such an alternative embodiment. By way of example, such an embodiment is constructed by spotting a series of dilutions of the biological antigen onto the nitrocellulose membrane element 10 with the highest concentration located at the first point 10a of the nitrocellulose membrane element 10 and the lowest concentration located at the second point 10b of the nitrocellulose membrane element 10 with sequential concentrations located therebetween at discrete sites. In the embodiments described above, the highest concentration of the biological antigen is located at the first end 10a of the nitrocellulose membrane element 10 and the lowest concentration is located at the second end 10b of the nitrocellulose membrane element 10. However, in an alternative embodiment, the reverse is applicable and the lowest concentration of the biological antigen is located at the first end 10a of the nitrocellulose membrane element 10 and the highest concentration is located at the second end 10b of the nitrocellulose membrane element 10.

In one embodiment, a control 40 is immobilised towards the first point 10a on the nitrocellulose membrane element 10. In an alternative embodiment, the control 40 is immobilised in a position separate from the first point 10a on the nitrocellulose membrane element 10 so as to avoid any confusion in the reading thereof. In one embodiment, the control is immobilised over an area that is substantially circular in shape. In an alternative embodiment, the control is immobilised over an area that is an alternative shape and/or position on the nitrocellulose membrane element 10. In a preferred embodiment, the control comprises a positive control. In one embodiment, the positive control is an anti-IgG antibody, preferably an anti-human IgG antibody. In an alternative embodiment, a control other than an anti-IgG antibody is immobilised on the nitrocellulose membrane element 10.

Use of the diagnostic device of FIG. 14 will now be described, in which the nitrocellulose membrane element 10 comprises a biological antigen immobilised thereon at a plurality of different concentrations. In the case that the biological sample contains the antibody, a greater amount of antibody binds to the antigen immobilised at the highest concentration (i.e. at the first point 10a on the nitrocellulose membrane element 10) compared to that immobilised at the lower concentrations (i.e. towards the second point 10b of the nitrocellulose membrane element 10). In turn, an approximately proportional amount of the molecular conjugate of the detection reagent binds to the antibody. The concentration of the gold particles from the molecular conjugate on the nitrocellulose membrane element 10 is thus highest at the first point 10a on the nitrocellulose membrane portion and results in a stronger detectable signal becoming visible on the nitrocellulose membrane element 10 within the aperture 12. The concentration of gold particles from the molecular conjugate is sequentially lower with proximity to the second point 10b of the nitrocellulose membrane element 10, owing to there being a reduced amount of antibody available for binding. The detectable signal is thus strongest at the first point 10a and weakens (e.g. visibly fades) in the direction of the second point 10b on the nitrocellulose membrane element. In one embodiment, the position between the first and second points 10a, 10b on the nitrocellulose membrane 10 at which the indicator is no longer visible is compared with a series of graduation marks 41 that are aligned alongside the second side portion 12d of the aperture 12. This enables a semi-quantitative readout of the level of antibody in the biological sample. In one embodiment, a series of biological samples are taken from an individual over time and each is tested on a diagnostic device 1 for comparison of the antibody level in the individual over time.

Referring to FIGS. 21A and 21B, variants of the embodiment shown in FIG. 14 are described, in which like components are shown with like reference numerals. A testing portion 11 comprises a lozenge-shaped area, centred within which is a lozenge-shaped aperture 12 defined by a first and a second end portion 12a, 12b and a first and a second side portion 12c, 12d. The testing portion 11 is convex towards a nitrocellulose membrane element 10. A series of graduation marks 41 are aligned with and extend alongside the second side portion 12d that defines the aperture 12. In alternative embodiments, the area of the testing portion 11 and/or the aperture 12 is an alternative shape such as being rectangular, circular, square et cetera. Referring to FIGS. 21A and 21B, the graduation marks 41 correspond to a numerical scale that increases in value as the graduation marks extend from the first end portion 12a to the second end portion 12b.

Referring to FIG. 21A, the nitrocellulose membrane element 10 comprises a biological antigen immobilised thereon at a plurality of different concentrations. The biological antigen is one or more as described above. The biological antigen is immobilised in a series of discrete lines 42, each of which extends in a substantially perpendicular manner from the second side portion 12d. A first discrete line 42a, located towards the first end portion 12a, comprises the biological antigen immobilised at a highest concentration and a second discrete line 42b, located towards the second end portion 12b, comprises the biological antigen immobilised at a lowest concentration, with the series of discrete lines therebetween having sequential concentration of biological antigen immobilised thereon. A control 40 comprising an anti-IgG antibody immobilised at a further discrete line is positioned on the nitrocellulose membrane element 10 between the first end portion 12a and the first discrete line 42a of the immobilised biological antigen. However, in alternative embodiments, the control is positioned in an alternative location on the nitrocellulose membrane element 10. As described above in relation to FIG. 14, the control is preferably a positive control and in certain embodiments comprises an alternative antibody or moiety immobilised thereon.

Referring to FIG. 21B, this embodiment comprises substantially the same components of that shown in FIG. 21A but further comprises a plurality of different concentrations of the biological antigen which extend between a first and a second point 10a, 10b on the nitrocellulose membrane element 10 in a substantially continuous gradation of their respective concentrations (as described in relation to FIG. 14). By way of example, such an embodiment is constructed by spraying the biological antigen onto the nitrocellulose membrane element 10 between the first and second points 10a, 10b, such that the highest concentration of the antigen is immobilised at the first point 10a and sequentially less antigen is immobilised as the second point 10b is approached. In one embodiment, the nitrocellulose membrane element 10 comprises the substantially continuous graduation of the respective concentrations of the biological antigen in the absence of the discrete lines 42.

In the embodiments shown in FIGS. 21A and 21B, the highest concentration of the biological antigen is immobilised on the biological membrane element 10 towards the first end portion 12a and the lowest concentration is located towards the second end portion 12b. However, in an alternative embodiment, the positioning of the concentrations is reversed such that the lowest concentration of the biological antigen is immobilised on the biological membrane element 10 towards the first end portion 12a and the highest concentration is located towards the second end portion 12b

In use of the variant embodiments shown in FIGS. 21A and 21B, a liquid, biological sample is applied to the nitrocellulose membrane element 10. In the case that the biological sample contains antibody specific for the biological antigen immobilised on the nitrocellulose membrane element 10, a greater amount of antibody binds to the antigen immobilised at the highest concentration (i.e. towards the first end portion 12a) compared to that immobilised at the lower concentrations (i.e. towards the second end portion 12b). In turn, an approximately proportional amount of the molecular conjugate of the detection reagent binds to the antibody. The concentration of the gold particles from the molecular conjugate on the nitrocellulose membrane element 10 is thus highest at the first discrete line 42a (and the first point 10a on the nitrocellulose membrane element 10 as shown in the embodiment of FIG. 21B) and results in a stronger detectable signal becoming visible on the nitrocellulose membrane element 10 within the aperture 12. The concentration of gold particles from the molecular conjugate is sequentially lower with proximity to the second end portion 12b, owing to there being a reduced amount of antibody available for binding. The detectable signal is thus strongest at the first discrete line 42a and becomes sequentially weaker in the direction of the second discrete line 42b (similarly, the detectable signal is strongest at the first point 10a and weakens in the direction of the second point 10b on the nitrocellulose membrane element 10 of the embodiment shown in FIG. 21B). The particular discrete line (and/or the position on the nitrocellulose membrane element 10 in the embodiment shown in FIG. 21B) at which the detectable signal is no longer visible is compared with the series of graduation marks 41 that are aligned alongside the second side portion 12d of the aperture 12. This enables a semi-quantitative readout of the level of antibody in the biological sample. By way of example, if a sample comprises a higher level of antibody then the detectable signal is visible at a position further towards the second end portion 12b (where a lower concentration of biological antigen is immobilised) than would be visible for a sample comprising a lower level of antibody. In one embodiment, a series of biological samples are taken from an individual over time and each is tested on a diagnostic device 1 for comparison of the antibody level in the individual over time.

It is to be understood that in certain embodiment, a single testing section 11 is provided on the diagnostic device 1, whereas in alternative embodiments, a plurality of testing sections 11 are provided. In one embodiment, the plurality of testing sections 11 are arranged in a grid on the diagnostic device 1. The grid arrangement enables a plurality of independent testing sections 11 to be provided on the diagnostic device 1. In one embodiment, each of the plurality of testing sections 11 has a respective aperture 12 and an associated section of the nitrocellulose membrane thereunder. Each of the respective sections of the nitrocellulose membranes has a different biological antigen immobilised thereon (or a variant thereof). It is to be appreciated that such an embodiment permits the rapid testing for the presence of antibodies specific for each of the different biological antigens, in one or a plurality of liquid, biological sample, in a controlled manner.

In a variant of the embodiments described above (such as those in which the biological antigen comprises a spike protein, or fragment thereof, of COVID-19 and those shown in FIGS. 13 and 14), the biological antigen is immobilised on a nitrocellulose membrane element 10 within an alternative diagnostic device. In one embodiment, the alternative diagnostic device is a lateral flow device. In such embodiments, it is to be understood that the biological antigen is immobilisable on the nitrocellulose membrane element 10. In one embodiment, the lateral flow device comprises a biological antigen, conjugated to a detectable moiety, present on a test strip. The biological antigen is capable of interacting with a first antibody if present in a liquid, biological sample. The test strip comprises a detection portion (such as a nitrocellulose membrane element 10) comprising a second antibody immobilised thereon, either directly or indirectly. The second antibody is capable of interacting with the first antibody. In one embodiment, second antibody is an anti-IgG, anti-IgM or anti-IgA antibody but it is not limited thereto. In use, a liquid, biological sample is applied to the test strip. If the liquid, biological sample comprises the first antibody specific for the biological antigen, it binds thereto. The biological antigen-first antibody complex is transported to the detection portion, wherein the first antibody is captured by the second antibody immobilised on the detection portion. That is to say, if the first antibody is an IgG antibody, it is captured by the second anti-IgG antibody. Thus the first antibody forms a bridge between the second antibody and the biological antigen such that all three components are immobilised on the detection portion, resulting in a concentration of the detectable moiety and a positive signal. In such embodiments, it is to be understood that the biological antigen is immobilised on the detection portion only in the presence of the first antibody, which in turn interacts with the second antibody immobilised on the detection portion.

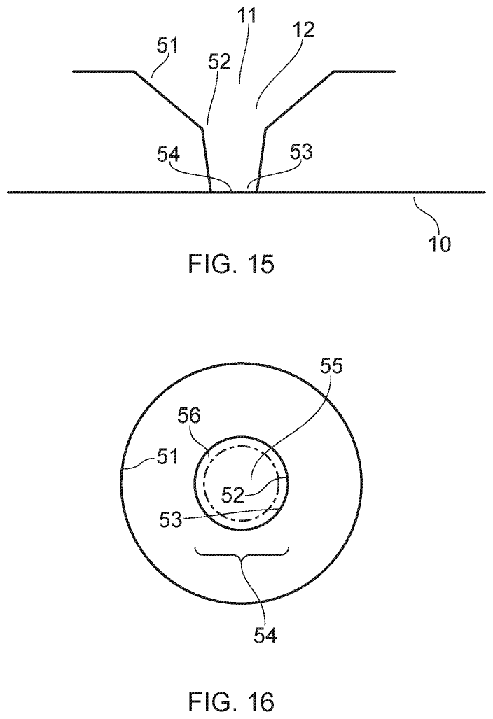

Referring, now, to FIGS. 15 and 16, a further embodiment of the present invention will now be described. The embodiment of FIGS. 15 and 16 is substantially the same as the embodiment of FIG. 1 and like components are shown with like reference numerals. In FIG. 15 a portion of a diagnostic device is shown in cross-section. In FIG. 16, a portion of the diagnostic device is shown from above. Thus there is provided a nitrocellulose membrane element 10 located beneath a testing portion 11, which contains an aperture 12. More specifically, the testing portion 11 is composed of a relatively wide, circular inlet 51 which tapers inwardly at a shallow gradient from the exterior towards the interior. At a neck 52, the gradient of the testing portion 11 changes to a relatively steep gradient from the exterior towards a relative narrow outlet 53.

As such, the section of the testing portion 11 which forms the outlet 53 contacts the nitrocellulose membrane element 10 and defines a circular, visible portion 54 of the nitrocellulose membrane element 10, which is to say the portion of the nitrocellulose membrane element 10 which can be seen through the aperture 12. The nitrocellulose membrane element 10 is bonded to the outlet 53 of the testing portion 11 by way of a liquid-tight seal around the perimeter of the outlet 53.

Located on the visible portion 54 is a circular detection portion 55 which is defined by the presence of a biological antigen (not shown) which is immobilised on the nitrocellulose membrane element 10 within the detection portion 55. Arranged around the perimeter of the detection portion 55 is a ring-shaped control portion 56 on which the biological antigen is not present.

It is to be understood that the visible portion 54 thus consists of the detection portion 55 and the ring-shaped control portion 56. Furthermore, it is to be noted that the surface area of the detection portion 55 (which can most readily be observed in FIG. 16) forms 95% of the surface area of the visible portion 54, with the remaining 5% of the surface area of the visible portion 54 consisting of the control portion 56. In alternative embodiments, the surface area of the detection portion 55 forms between 60% and 99%, preferably between 70% and 99%, between 80% and 99%, between 80% and 99% or between 95% and 99% of the surface area of the visible portion 54 with the balance of the surface area being formed by the control portion 56. Nevertheless, in all embodiments, the control portion 56 is present. As mentioned above, the relative size of the control portion is variable from embodiment to embodiment but what is important is that the surface area of the control portion 56 is large enough to be visible.

The diagnostic device of the embodiment of FIGS. 15 and 16 is used in a similar manner as the diagnostic device of the embodiment of FIG. 1. In summary, a biological sample is deposited via the aperture 12 on the nitrocellulose membrane element 10 and, more specifically, on the visible portion 54 thereof. It is to be understood that in this embodiment, the volume of the biological sample may be low. Nevertheless, because of the tapering cross-section of the testing portion 11 from the relatively wide inlet 51 to the relatively narrow outlet 53, the biological sample is concentrated on the visible portion 54. Furthermore, even though the volume of the biological sample may be low, because almost all of the visible portion 54 is composed of the detection portion 55 on which the biological antigen is present, almost all of the biological sample comes into contact with the biological antigen. Antibody in the biological sample that is capable of doing so binds to the biological antigen. Subsequently, a detection reagent (as described in the embodiment of FIG. 1) is deposited on the visible portion 54 and binds to antibody that has been immobilised on the detection portion 55. It is to be understood that the control portion 56, while relatively small, is nonetheless visible and is not coloured by the detection reagent, thereby permitting a visual comparison between the detection portion 55 and the visible portion 54. It is to be appreciated that in principle any volume of biological sample may be detected in this embodiment so long as a visible comparison between the detection portion 55 and the control portion 56 is possible.

In the embodiment shown in FIGS. 15 and 16, the region of the testing portion 11 between the neck 52 and the outlet 53 is shown with a steep gradient. However, it is to be appreciated that in variants of this embodiment, this region is perpendicular to the upper surface of the nitrocellulose membrane element 10.

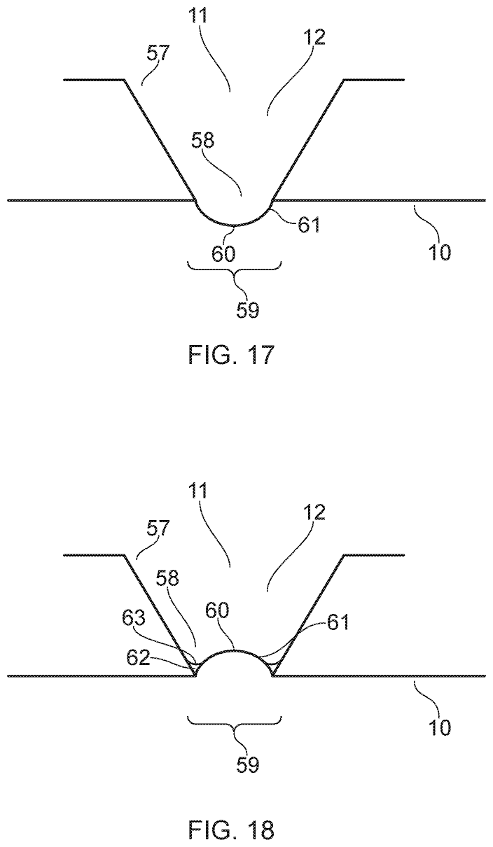

Referring, now, to FIG. 17, a further embodiment of the present invention will now be described. The embodiment of FIG. 17 is substantially the same as the embodiment of FIG. 1 and like components are shown with like reference numerals. Thus there is provided a nitrocellulose membrane element 10 beneath a testing portion 11 which contains an aperture 12. More specifically, the testing portion 11 is composed of an inlet 57 having a relatively wide cross-section area which tapers to an outlet 58 which has a relatively narrow cross-sectional area.

As such, the section of the testing portion 11 which forms the outlet 58 contacts the nitrocellulose membrane element 10 and defines a circular, visible portion 59 of the nitrocellulose membrane element 10, which is to say the portion of the nitrocellulose membrane element 10 which can be seen through the aperture 12. The nitrocellulose membrane element 10 is bonded to the outlet 58 of the testing portion 11 by way of a liquid-tight seal around the perimeter of the outlet 58.

Located on the visible portion 59 is a circular detection portion 60 which is defined by the presence of a biological antigen (not shown) which is immobilised on the nitrocellulose membrane element 10 within the detection portion 60. Arranged around the perimeter of the detection portion 60 is a ring-shaped control portion 61 on which the biological antigen is not present. In this embodiment, rather than the visible portion 59 having a planar upper surface, as in previous embodiments, the upper surface of the visible portion 59 is in the shape of a concave depression, the outer perimeter of which corresponds to the perimeter of the outlet 58 of the testing portion 11. As such, the detection portion 60 forms the bottom of the concave depression and is, itself, in the shape of a concave depression. Importantly, this means that the detection portion 60 is lower than the control portion 61.

The embodiment of FIG. 17 is used in substantially the same manner as in the previous embodiments. To summarise, a liquid biological sample is deposited in the testing portion 11 and passes through the aperture 12 by way of the inlet 57 and then the outlet 58 at which point it comes into contact with the detection portion 60. Because the detection portion 60 is in the shape of a concave depression, the biological sample coalesces at the bottom of the detection portion 60. Therefore, even if the volume of the biological sample is low, it is concentrated on the detection portion 60 on which the biological antigen is immobilised and therefore ensures that all of the volume of such a biological sample comes into contact with the biological antigen. Furthermore, in this embodiment, the run-off of biological sample via the control portion 61 without coming into contact with the detection portion 60 is avoided. A detection reagent is then added to the testing portion 11 and the results are visualised as in the previous embodiments. In this way, this embodiment is particularly useful for the detection of antibodies in a low volume of biological sample.

Referring to FIG. 18, a further embodiment of the present invention will now be described. The embodiment of FIG. 18 is substantially the same as the embodiment of FIG. 17 and like components are shown with like reference numerals. However, in the embodiment of FIG. 18, rather than the visible portion 59 of the porous membrane element 10 being in the shape of a concave depression, it is instead in the shape of a convex dome. As such, the detection portion 60 forms the top of the convex dome and is, itself, in the shape of a convex dome. Importantly, this means that the detection portion 60 is higher than the control portion 61. Furthermore, an annular channel 62 is provided between the side wall of the visible portion 59 and the inner side of the outlet 58 of the testing portion 11.

The embodiment of FIG. 18 is used in substantially the same manner as in the previous embodiments. To summarise, a liquid biological sample is deposited in the testing portion 11 and passes through the aperture 12 by way of the inlet 57 and then the outlet 58 at which point it comes into contact with the detection portion 60. However, in this embodiment, the biological sample, whilst coming into contact with the biological antigen which is immobilised on the detection portion, has a tendency to run off the top of the detection portion 60 and into the annular channel 62 and form a pool 63 therein, under the force of gravity and the surface tension of the biological sample. Due to this arrangement, any visual contaminants in the sample are urged into the annular channel 62 and do not impair visualisation of the detection portion 60 by the user. Such visual contaminants include particulate matter such as the results of haemolysis.

In preferred embodiments, a kit is provided comprising a diagnostic device 1, as described above, and a supply of the molecular conjugate in a suitable liquid medium.

In the embodiments described above, the molecular conjugate has been described as comprising an antibody conjugated to a gold particle. However, in alternative embodiments, the respective components may be substituted for alternative moieties having a similar activity. For example, instead of a gold particle, another detectable moiety such as a platinum particle or a dye molecule is provided in some embodiments. Alternatively, in some embodiments, instead of a gold particle, a fluorescent marker molecule is the detectable moiety which is visualised under UV light. As a further alternative to a gold particle, a magnetised marker, an electrically conductive marker, a nanoparticle, a radio-frequency identification (RFID)-tag or an exothermic marker is provided in some embodiments. Instead of an antibody, an alternative anti-IgG or anti-IgM binding moiety is provided in some embodiments.

In the above described embodiments, a biological antigen is immobilised on the nitrocellulose membrane element 10. The detection reagent comprises an anti-IgG or anti-IgM binding moiety. However, it is to be appreciated that the present invention is not limited to this arrangement. For example, in one embodiment, instead of a biological antigen being immobilised on the nitrocellulose membrane element 10, an antibody specific for an antigen, virus or protein is immobilised on the nitrocellulose membrane element 10 and the device is for the detection of the antigen, virus or protein. In one specific embodiment, a first anti-COVID-19 antibody is immobilised on the nitrocellulose membrane element 10 and the binding moiety of the molecular conjugate in the detection reagent is a second anti-COVID-19 antibody. In some variants of this embodiment, the second anti-COVID-19 antibody is conjugated to a detectable moiety such as a gold particle. In these variants, after the liquid, biological sample is applied to the exposed section of the nitrocellulose membrane element 10, the detection reagent is also applied to the nitrocellulose membrane element 10. If the liquid biological sample contains COVID-19 particles then these particles bind to the first anti-COVID-19 antibody and are immobilised on the nitrocellulose membrane element 10 and subsequently the second anti-COVID-19 antibody also binds to the virus particles such that the virus particles form a bridge between the first and second anti-COVID-19 antibodies thus resulting in a concentration of the detectable moiety and a positive signal.

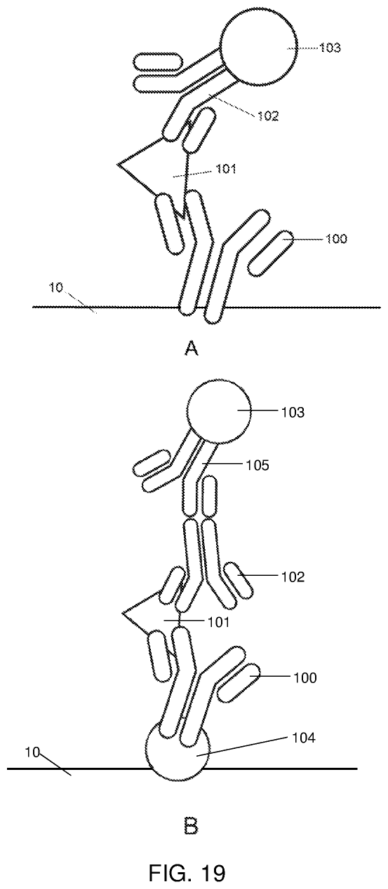

FIG. 19A depicts an example of such an embodiment. Referring to FIG. 19A, a first anti-COVID-19 antibody 100 is immobilised on a nitrocellulose membrane element 10. In one embodiment, the first anti-COVID-19 antibody 100 is immobilised on the nitrocellulose membrane element 10 directly. In an alternative embodiment, the first anti-COVID-19 antibody 100 is immobilised on the nitrocellulose membrane element 10 via a linking moiety (not shown in FIG. 19A). The linking moiety binds to both the first anti-COVID-19 antibody 100 and the nitrocellulose membrane element 10 and thus immobilises the first anti-COVID-19 antibody 100 on the nitrocellulose membrane element 10. In one embodiment, the linking moiety is a protein. In one embodiment, the linking moiety promotes the uniform immobilisation of the first anti-COVID-19 antibody 100 across the surface of the nitrocellulose membrane element 10 and/or facilitates the appropriate orientation of the first anti-COVID-19 antibody 100 on the nitrocellulose membrane 10. The first anti-COVID-19 antibody 100 is capable of binding COVID-19 particles (antigen) 101 present in a liquid, biological sample. In FIG. 19A, a detection reagent has been applied which comprises a molecular conjugate comprising a binding moiety which is a second anti-COVID-19 antibody 102 and a detectable moiety 103. That is to say, the second anti-COVID-19 antibody 102 is conjugated to the detectable moiety 103. In one embodiment, the detectable moiety 103 is a gold particle but as described above, it is not limited thereto.

In use, after the liquid, biological sample is applied to the exposed section of the nitrocellulose membrane element 10, the detection reagent is also applied to the nitrocellulose membrane element 10. If the liquid, biological sample contains COVID-19 particles (antigen) 101 then these particles (antigen) 101 bind to the first anti-COVID-19 antibody 100 and are immobilised on the nitrocellulose membrane element 10 and subsequently the second anti-COVID-19 antibody 102 also binds to the virus particles (antigen) 101 such that the virus particles (antigen) 101 form a bridge between the first and second anti-COVID-19 antibodies 100, 102 thus resulting in a concentration of the detectable moiety 103 and a positive signal.

In other variants of this embodiment, the second anti-COVID-19 antibody is not conjugated to a detectable moiety itself and instead a reporter antibody is added after the addition of the detectable reagent in order to give rise to a detectable signal.

FIG. 19B depicts an example of such an embodiment. Referring to FIG. 19B, a first anti-COVID-19 antibody 100 is immobilised on a nitrocellulose membrane element 10. In one embodiment, the first anti-COVID-19 antibody 100 is immobilised on the nitrocellulose membrane element 10 via a linking moiety 104. The linking moiety 104 binds to both the first anti-COVID-19 antibody 100 and the nitrocellulose membrane element 10 and thus immobilises the first anti-COVID-19 antibody 100 on the nitrocellulose membrane element 10. In one embodiment, the linking moiety 104 is a protein. In one embodiment, the linking moiety 104 promotes the uniform immobilisation of the first anti-COVID-19 antibody 100 across the surface of the nitrocellulose membrane element 10 and/or facilitates the appropriate orientation of the first anti-COVID-19 antibody 100 on the nitrocellulose membrane 10. In an alternative embodiment, the first anti-COVID-19 antibody 100 is immobilised on the nitrocellulose membrane element 10 directly (not shown in FIG. 19B). The first anti-COVID-19 antibody 100 is capable of binding COVID-19 particles (antigen) 101 present in a liquid, biological sample. In FIG. 19B, a second anti-COVID-19 antibody 102 is added which is not conjugated to a detectable moiety 103 itself. Instead, a reporter antibody 105 is further added which is conjugated to the detectable moiety 103. In one embodiment, the detectable moiety 103 is a gold particle but as described above, it is not limited thereto. The reporter antibody 105 is capable of binding to the second anti-COVID-19 antibody 102. In one embodiment, the second anti-COVID-19 antibody 102 is a human antibody and the reporter antibody 105 is an anti-human antibody. However, in alternative embodiments, the second anti-COVID-19 antibody 102 and the reporter antibody 105 are an alternative complementary pair, such as a mouse and anti-mouse antibody respectively.

In use, after the liquid, biological sample is applied to the exposed section of the nitrocellulose membrane element 10, the second anti-COVID-19 antibody 102 is added to the nitrocellulose membrane element 10. If the liquid, biological sample contains COVID-19 particles (antigen) 101 then these particles (antigen) 101 bind to the first anti-COVID-19 antibody 100 and are immobilised on the nitrocellulose membrane element 10 and subsequently the second anti-COVID-19 antibody 102 also binds to the virus particles (antigen) 101 such that the virus particles (antigen) 101 form a bridge between the first and second anti-COVID-19 antibodies 100, 102. The reporter antibody 105 is then added to the nitrocellulose membrane element 10 and binds to the second anti-COVID-19 antibody 102 (which is itself bound to the virus particles (antigen) 101) thus resulting in a concentration of the detectable moiety 103 and a positive signal. In the this embodiment, the reporter antibody 105 is added subsequent to the addition of the second anti-COVID-19 antibody 102. In an alternative embodiment, the reporter antibody 105 and the second anti-COVID-19 antibody 102 are added substantially simultaneously to the nitrocellulose membrane element 10.

In either of these variants (for example, as described in relation to FIGS. 19A and 19B, the detection of virus particles in the liquid, biological sample is indicative of a current infection in the individual from whom the sample has been obtained.

It is preferred that the COVID-19 particles (antigen) 101 comprise COVID-19 spike protein, preferably COVID-19 S1 and/or S2 spike protein. In such an embodiment, the first and second anti-COVID-19 antibodies 100, 102, which are capable of binding the COVID-19 particles (antigen) 101, are each a COVID-19 spike antibody, preferably an anti-COVID-19 S1 and/or S2 spike antibody. In some embodiments, the COVID-19 particles (antigen) 101 are comprised within a larger or a whole virus particle (not shown in FIGS. 19A and 19B). In a preferred embodiment, the nitrocellulose membrane element 10 comprises pores (not shown in FIGS. 19A and 19B) which are of a diameter slightly larger than that of the target virus (e.g. SARS-CoV-2) thus permitting unbound whole virus that is present in the liquid, biological sample to pass through.

The present invention has predominantly been described in relation to antigen-antibody binding pairs, in particular, where the biological antigen comprises a Coronavirus protein or a fragment thereof. It is to be understood that in some embodiments, the biological antigen comprises a protein or fragment thereof derived from a virus, wherein the virus is one other than a Coronavirus. In one embodiment, the virus is a virus that infects a plant. In one embodiment, the virus is a virus that infects a human, a non-human animal such as a primate or a non-mammalian species such as a fish. In other embodiments, the biological antigen is a product (such as a protein) from an animal (e.g. mammal or fish), bacteria, plant or fungus (e.g. a mould).

It is also to be understood that the present invention is not limited to reporter assays which involve an antigen-antibody binding pair and can be applied to any assay involving a reporter-analyte pair. Such reporter-analyte pairs can involve molecules which specifically bind each other (e.g. a nucleic acid molecule and a protein), molecules which have other specific interactions with each other (e.g. an enzyme and a substrate molecule), a toxin and a target, a hormone and a receptor etc. Therefore, in yet further embodiments of the present invention, the biological antigen of the above described embodiments is replaced with one member of the reporter-analyte pair and the binding moiety of the detection reagent is replaced with the other member of the reporter-analyte pair.

It is to be appreciated that in some alternative embodiments, the diagnostic device 1 is supplied without the provision of one member of a reporter-analyte pair immobilised on the nitrocellulose membrane element 10 such that the end-user may add the desired member of the reporter-analyte pair.

In the above description, the device has been described as a “diagnostic device”. However, is to be appreciated that the device may be used to test for immunity (e.g. by vaccination), infection or past exposure by analysis of samples from an individual and so may alternatively be described as a “detection device”.

### EXAMPLES

Hereinafter, the invention will be specifically described with reference to an Example. However, this Example does not limit the technical scope of the invention.

### Example 1: A Diagnostic Device Comprising the S1 and S2 Spike Proteins Exhibits High Specificity and Sensitivity

A diagnostic device according to the present invention and comprising the S1 spike protein and the S2 spike protein (an edited version thereof in which the C-terminal 6 or 62 amino acids were removed) immobilised on a nitrocellulose membrane was used within the UK NEQAS benchmarking exercise for SARS-CoV-2/COVID-19 antibody detection. The S1 and S2 spike protein were produced in insect cells. In brief, samples that had been independently verified as being positive or negative for COVID-19 antibodies were tested using the device of the present invention.

The device of the present invention indicated that 76 of the samples were positive and that 186 of the samples were negative. This corresponded to a sensitivity of 100% and a specificity of 99.5%. Furthermore, the specificity may, in fact, be higher owing to the re-classification of one of the samples.

In conclusion, a diagnostic device comprising the S1 and S2 spike proteins demonstrated a high level of sensitivity and specificity which is in contrast with devices that incorporate or rely on the COVID-19 nucleoprotein.

### Schedule of Sequence Listing

SEQ ID NO: 1: amino acid sequence of SARS-CoV-2 S1 subunit including predicted cleavage sites (FIG. 9, shown in underline)

SEQ ID NO: 2: amino acid sequence of SARS-CoV-2 spike protein (FIG. 9).

SEQ ID NO: 3: amino acid sequence of SARS-CoV-2 receptor binding domain (FIG. 10, shown in underline).

SEQ ID NO: 4: amino acid sequence of SARS-CoV-2 S1 subunit (FIG. 10).

SEQ ID NO: 5: amino acid sequence of SARS-CoV-2 S2 subunit (FIG. 11A).

SEQ ID NO: 6: amino acid sequence of SARS-CoV-2 S2′ subunit (FIG. 11B).

SEQ ID NO: 7: amino acid sequence of SARS-CoV-2 nucleoprotein (FIG. 12).

1. A diagnostic device for detecting a first member of a reporter-analyte pair or a first member of each of a plurality of reporter-analyte pairs comprising:
an inlet for receiving a liquid, biological sample; and
a porous membrane element comprising a detection portion, the detection portion being in liquid communication with the inlet and a second member of the or each reporter-analyte pair being immobilisable on the detection portion,
wherein one of the first or second member of the or each reporter-analyte pair comprises a biological antigen and the other of the first or second member of the or each reporter-analyte pair comprises an antibody specific for the biological antigen, and
wherein the biological antigen comprises:
(a) a COVID-19 S1 spike protein comprising the sequence of SEQ ID NO: 4 (as represented in FIG. 10) or a sequence having at least 99% or 100% sequence identity to the sequence of SEQ ID NO: 4 (as represented in FIG. 10); and/or
(b) a first biological antigen from a first reporter-analyte pair comprising a COVID-19 S1 spike protein or a fragment thereof, and
a second biological antigen from a second reporter-analyte pair comprising a COVID-19 S2 spike protein or a fragment thereof,
and wherein the device is for independent detection of a spike protein, or a fragment thereof, or of an antibody specific for a spike protein, or a fragment thereof, in the biological sample.

2. The diagnostic device according to claim 1, wherein the first biological antigen comprising the COVID-19 S1 spike protein or the fragment thereof is a polypeptide comprising a sequence of at least 8, 10, 12, 14, 16, 18, 20, 30, 100, 200 or 300 amino acids from an amino acid sequence having at least 70%, 80%, 90%, 95%, 99% or 100% sequence identity to the sequence of SEQ ID NO: 4 (as represented in FIG. 10), optionally wherein the COVID-19 S1 spike protein or the fragment thereof comprises the COVID-19 S1 spike protein receptor binding domain having the sequence of SEQ ID NO: 3.

3. The diagnostic device according to claim 1 or 2, wherein the second biological antigen comprising the COVID-19 S2 spike protein or the fragment thereof is a polypeptide comprising a sequence of at least 8, 10, 12, 14, 16, 18, 20, 30, 100, 200 or 300 amino acids from an amino acid sequence having at least 70%, 80%, 90%, 95%, 99% or 100% sequence identity to the sequence of SEQ ID NO: 5 (as represented in FIG. 11A) or SEQ ID NO: 6 (as represented in FIG. 11B).

4. The diagnostic device according to any one of claims 1 to 3, wherein the COVID-19 S2 spike protein or the fragment thereof comprises a sequence in which the C-terminal six or 62 amino acids are deleted from the sequence of SEQ ID NO: 5 (as represented in FIG. 11A) or SEQ ID NO: 6 (as represented in FIG. 11B).

5. The diagnostic device according to any one of claims 1 to 4, wherein the COVID-19 S1 spike protein or the fragment thereof and the COVID-19 S2 spike protein or the fragment thereof are comprised within a single polypeptide chain, the single polypeptide chain being immobilisable on the detection portion.

6. The diagnostic device according to any one of claims 1 to 5, wherein the biological antigen further comprises a third biological antigen from a third reporter-analyte pair and wherein the third biological antigen comprises a COVID-19 nucleoprotein or a fragment thereof, preferably comprises the sequence of SEQ ID NO: 7 (as represented in FIG. 12).

7. The diagnostic device according to any one of claims 1 to 6, wherein the porous membrane element further comprises a reference element for indicating a level of the first member of the or each reporter-analyte pair in the liquid, biological sample, preferably wherein the reference element is a plurality of reference elements.

8. The diagnostic device according to any one of claims 1 to 7, wherein the second member of the or each reporter-analyte pair is immobilised on the detection portion.

9. A kit comprising a diagnostic device according to any one of the preceding claims and a detection reagent, the detection reagent comprising a molecular conjugate which comprises a detectable moiety bound to a moiety capable of binding the first member of the reporter-analyte pair.

10. A kit according to claim 9 wherein the moiety capable of binding the first member of the reporter-analyte pair is an antibody.

11. A kit according to claim 10 wherein the molecular conjugate comprises a gold particle conjugated to an anti-IgG or anti-IgM antibody.

12. A method of testing for the presence of a first member of a reporter-analyte pair in a liquid, biological sample, comprising the steps of:
I) providing a diagnostic device according to any one of claims 1 to 8;
II) depositing the biological sample onto the porous membrane element such that the biological sample contacts the second member of the reporter-analyte pair immobilisable on the porous membrane element and such that first member of the reporter-analyte pair interacts with the second member of the reporter-analyte pair,
wherein an interaction between the first member of the reporter-analyte pair and the second member of the reporter-analyte pair is detectable.

13. The method according to claim 12, wherein the method further comprises the step of:
III) depositing a detection reagent onto the porous membrane element such that the detection reagent contacts the first member of the reporter-analyte pair, the detection reagent providing a detectable signal when contacting the first member of the reporter-analyte pair.

14. The method according to claim 13, wherein the detection reagent comprises a detectable moiety bound to a moiety capable of binding the first member of the reporter-analyte pair.

15. The method according to any one of claims 12 to 14, wherein the second member of the reporter-analyte pair comprises a biological antigen, the biological antigen comprising a spike protein, or a fragment thereof, of COVID-19 and wherein the first member of the reporter-analyte pair comprises an antibody specific for the spike protein, or the fragment thereof, of COVID-19.

16. The method according to any one of claims 12 to 15, wherein the diagnostic device provided is a diagnostic device according to any one of claims 1 to 8, wherein the reporter-analyte pair is a first and/or a second reporter-analyte pair,
wherein the second member of the first reporter-analyte pair is a COVID-19 S1 spike protein or a fragment thereof and the first member of the first reporter-analyte pair is an antibody specific for the COVID-19 S1 spike protein or a fragment thereof; and/or
wherein the second member of the second reporter-analyte pair is a COVID-19 S2 spike protein or a fragment thereof and the first member of the second reporter-analyte pair is an antibody specific for the COVID-19 S2 spike protein or a fragment thereof, and wherein the presence of an antibody specific for the COVID-19 S1 spike protein or the fragment thereof and/or the COVID-19 S2 spike protein or the fragment thereof in the biological sample indicates current or prior infection by or vaccination against COVID-19.

17. The method according to any one of claims 12 to 15, wherein the diagnostic device provided is a diagnostic device according to any one of claims 6 to 8, wherein the reporter-analyte pair is a first, a second and a third reporter-analyte pair,
wherein the second member of the first reporter-analyte pair is a COVID-19 S1 spike protein or a fragment thereof and the first member of the first reporter-analyte pair is an antibody specific for the COVID-19 S1 spike protein or a fragment thereof;
wherein the second member of the second reporter-analyte pair is a COVID-19 S2 spike protein or a fragment thereof and the first member of the second reporter-analyte pair is an antibody specific for the COVID-19 S2 spike protein or a fragment thereof; and
wherein the second member of the third reporter-analyte pair is a COVID-19 nucleoprotein or a fragment thereof and the first member of the third reporter-analyte pair is an antibody specific for the COVID-19 nucleoprotein or a fragment thereof, and wherein the presence of an antibody specific for the COVID-19 nucleoprotein, or the fragment thereof, and the absence of an antibody specific for the COVID-19 S1 spike protein, or the fragment thereof, and the absence of an antibody specific for the COVID-19 S2 spike protein, or the fragment thereof, indicates current or prior infection by or vaccination against a beta group coronavirus other than COVID-19.

18. A diagnostic device for detecting a first member of a reporter-analyte pair comprising:
a casing comprising a testing portion having an aperture therein, the aperture having an inlet for receiving a liquid, biological sample and an outlet for releasing the liquid, biological sample;
a porous membrane element comprising a detection portion, a second member of the reporter-analyte pair being immobilised on the detection portion, the outlet being in liquid communication with the detection portion via a detection flow path; and
a flow control structure for limiting or preventing the flow of the liquid, biological sample from the outlet along a bypass flow path, which bypasses the detection portion, instead of the detection flow path,
wherein the flow control structure comprises:
(a) a liquid-tight seal present between the testing portion and the porous membrane element, which prevents the liquid, biological sample from flowing along the bypass flow path which bypasses the detection portion; or
(b) an arrangement of gates or obstacles which are present in the bypass flow path and which limit the liquid, biological sample from flowing along the bypass flow path which bypasses the detection portion.

19. A diagnostic device according to claim 18, wherein the liquid-tight seal surrounds the aperture.

20. A diagnostic device according to claim 18 or 19 wherein the liquid-tight seal is an ultrasonic weld or other controlled seal between the testing portion and the porous membrane element.

21. A diagnostic device according to any one of claims 18 to 20 wherein the casing and the liquid-tight seal form a liquid-tight chamber around the porous membrane element from which only the detection portion of the porous membrane element is exposed from the chamber.

22. A diagnostic device according to any one of claims 18 to 21 wherein the porous membrane element is located adjacent to the testing portion such that the detection portion is visible through the inlet of the aperture.

23. A diagnostic device according to any one of claims 18 to 22, further comprising a liquid-absorbent pad in liquid-communication with the detection portion of the porous membrane element, the detection flow path being from the outlet of the aperture through the detection portion and into the liquid-absorbent pad.

24. A diagnostic device according to claim 23, wherein the bypass flow path is from the outlet of the aperture to the liquid-absorbent pad, without passing through the detection portion of the porous membrane element.

25. A diagnostic device according to claim 23 or 24 wherein the liquid-absorbent pad comprises an opening therein, and the porous membrane element is located between the liquid-absorbent pad and the aperture in the testing portion, the opening in the liquid-absorbent pad being aligned with the aperture in the testing portion.

26. A diagnostic device according to any one of claims 18 to 25 wherein the porous membrane element comprises a nitro-cellulose membrane.

27. A diagnostic device according to any one of claims 18 to 26 wherein the second member of the reporter-analyte pair comprises a biological antigen and the first member of the reporter-analyte pair comprises an antibody specific for the biological antigen.

28. A diagnostic device according to claim 27 wherein the biological antigen comprises a Coronavirus protein or fragment thereof.

29. A diagnostic device according to claim 28 wherein the coronavirus protein is a Covid-19 protein, preferably a COVID-19 S1 spike protein or fragment thereof.

30. A diagnostic device according to claim 29 wherein the coronavirus protein or fragment thereof is a polypeptide comprising a sequence of at least 8, 10, 12, 14, 16, 18, 20, 30, 100, 200 or 300 amino acids from an amino acid sequence having at least 70%, 80%, 90%, 95%, 99% or 100% sequence identity to the sequence of SEQ ID NO: 4 (as represented in FIG. 10).

31. A diagnostic device according to any one of claims 18 to 30 wherein the first member of the reporter-analyte pair comprises a biological antigen and the second member of the reporter-analyte pair comprises an antibody specific for the biological antigen.

32. A diagnostic device according to claim 31 wherein the biological antigen comprises a COVID-19 protein or fragment thereof.

33. A diagnostic device according to any one of claims 18 to 32 wherein the casing comprises at least one securing structure for locating the porous membrane element in a position covering the aperture.

34. A diagnostic device according to any one of claims 18 to 33, wherein the porous membrane element further comprises a reference element for indicating a level of the first member of the reporter-analyte pair in the liquid, biological sample, preferably wherein the reference element is a plurality of reference elements.

35. A kit comprising a diagnostic device according to any one of claims 18 to 34 and a detection create reagent, the detection reagent comprising a molecular conjugate which comprises a detectable moiety bound to a moiety capable of binding the first member of the reporter-analyte pair.

36. A kit according to claim 35 wherein the moiety capable of binding the first member of the reporter-analyte pair is an antibody.

37. A kit according to claim 36 wherein the molecular conjugate comprises a gold particle conjugated to an anti-IgG or anti-IgM antibody.

38. A method of testing for the presence of a first member of a reporter-analyte pair in a liquid, biological sample, comprising the steps of:
I) providing a diagnostic device according to any one of claims 18 to 34;
II) depositing the biological sample onto the porous membrane element via the aperture such that the biological sample contacts the second member of the reporter-analyte pair immobilised on the porous membrane element and such that first member of the reporter-analyte pair interacts with the second member of the reporter-analyte pair;
III) depositing a detection reagent onto the porous membrane element via the aperture such that the detection agent contacts the first member of the reporter-analyte pair, the detection reagent providing a detectable signal when contacting the first member of the reporter-analyte pair.

39. A method according to claim 38, wherein the detection reagent comprises a detectable moiety bound to a moiety capable of binding the first member of the reporter-analyte pair.

40. A diagnostic device for detecting a first member of a reporter-analyte pair comprising:
an inlet for receiving a liquid, biological sample;
a porous membrane element comprising a detection portion, the detection portion being in liquid communication with the inlet,
wherein the detection portion comprises a second member of the reporter-analyte pair and a variant of the second member of the reporter-analyte pair immobilisable thereon, and
wherein an interaction between the first member of the reporter-analyte pair and the second member of the reporter-analyte pair is separately detectable from an interaction between the first member of the reporter-analyte pair and the variant of the second member of the reporter-analyte pair.

41. The diagnostic device according to claim 40, wherein the second member of the reporter-analyte pair is immobilisable on the detection portion at a discrete site from the variant of the second member of the reporter-analyte pair.

42. The diagnostic device according to claim 40 or 41, further comprising a control immobilisable on the detection portion.

43. The diagnostic device according to any one of claims 40 to 42, wherein the second member of the reporter-analyte pair and/or the variant of the second member of the reporter-analyte pair and/or the control are immobilised on the detection portion.

44. The diagnostic device according to claim 43 as dependent on claim 41, wherein the control is positioned between the second member of the reporter-analyte pair and the variant of the second member of the reporter-analyte pair.

45. The diagnostic device according to any one of claims 40 to 44, wherein the second member of the reporter-analyte pair comprises a biological antigen and the first member of the reporter-analyte pair comprises an antibody specific for the biological antigen.

46. The diagnostic device according to claim 45, wherein the biological antigen is a protein, a polypeptide or a fragment thereof.

47. The diagnostic device according to any one of claims 40 to 46, wherein the second member of the reporter-analyte pair and the variant of the second member of the reporter-analyte pair are produced in a first and a second host cell respectively, wherein the first and the second host cell are each from a different species.

48. The diagnostic device according to any one of claims 40 to 47, wherein the second member of the reporter-analyte pair and the variant of the second member of the reporter-analyte pair comprise (a) and (b) below respectively:
(a) a biological antigen comprising a COVID-19 S1 spike protein or a fragment thereof, preferably wherein the COVID-19 S1 spike protein or the fragment thereof is a polypeptide comprising a sequence of at least 8, 10, 12, 14, 16, 18, 20, 30, 100, 200 or 300 amino acids from an amino acid sequence having at least 70%, 80%, 90%, 95%, 99% or 100% sequence identity to the sequence of SEQ ID NO: 4 (as represented in FIG. 10); and
(b) a biological antigen comprising a sequence having at least 70% sequence identity and less than 100% sequence identity to (a).

49. The diagnostic device according to any one of claims 40 to 48, wherein the variant of the second member of the reporter-analyte pair is a plurality of variants.

50. A diagnostic device for detecting a first member of a reporter-analyte pair or a first member of each of a plurality of reporter-analyte pairs comprising:
an inlet for receiving a liquid, biological sample; and
a porous membrane element comprising a detection portion, the detection portion being in liquid communication with the inlet and a second member of the or each reporter-analyte pair being immobilisable on the detection portion,
wherein one of the first or second member of the or each reporter-analyte pair comprises a biological antigen and the other of the first or second member of the or each reporter-analyte pair comprises an antibody specific for the biological antigen,
wherein the biological antigen comprises a COVID-19 S1 spike protein or a fragment thereof comprising a sequence in which one or more amino acids are substituted in a sequence consisting of the N-terminal 100, 200, 300, 400, 500 or 600 amino acids of the sequence of SEQ ID NO: 4 (as represented in FIG. 10), and
wherein the device is for independent detection of the COVID-19 S1 spike protein, or the fragment thereof, in which one or more amino acids has been substituted, or of an antibody specific for the COVID-19 S1 spike protein, or the fragment thereof, in which one or more amino acids has been substituted, in the biological sample.

51. A diagnostic device for detecting a first member of a reporter-analyte pair comprising:
an inlet for receiving a liquid, biological sample,
a porous membrane element comprising a detection portion, the detection portion being in liquid communication with the inlet,
wherein the detection portion comprises a second member of the reporter-analyte pair immobilised thereon at a plurality of different concentrations, and wherein the plurality of different concentrations of the second member of the report-analyte pair extend between a first point on the detection portion, at which the concentration of the second member of the reporter-analyte pair is highest, through to a second point on the detection portion, at which the concentration of the second member of the reporter-analyte pair is lowest and at sequential concentrations therebetween.

52. The diagnostic device according to claim 51, wherein the plurality of different concentrations of the second member of the reporter-analyte pair extend between the first point and the second point in a substantially continuous gradation of their respective concentrations.

53. The diagnostic device according to claim 51, wherein the plurality of different concentrations of the second member of the reporter-analyte pair extend between the first point and the second point at a series of discrete sites.

54. The diagnostic device according to any one of claims 51 to 53, further comprising a control.

55. The diagnostic device according to any one of claims 40 to 54, wherein the porous membrane element further comprises a reference element for indicating a level of the first member of the reporter-analyte pair in the liquid, biological sample, preferably wherein the reference element is a plurality of reference elements.

56. A diagnostic device for detecting a first member of a reporter-analyte pair comprising:
a casing comprising a testing portion having an aperture therein, the testing portion having an inlet for receiving a liquid, biological sample and an outlet for releasing the liquid, biological sample;
a membrane element having a detection portion on which a second member of the reporter-analyte pair is immobilised, the outlet being in liquid communication with the detection portion via a detection flow path;
the outlet of the testing portion contacting the membrane element and defining a visible portion of the membrane element, the visible portion of the membrane element comprising the detection portion,
and wherein the surface area of the detection portion is at least 60% but less than 100% of the surface area of the visible portion.

57. A diagnostic device according to claim 56 wherein the membrane element is a porous membrane element.

58. A diagnostic device according to claim 56 or 57 wherein the surface area of the detection portion is between 90% and 95% of the surface area of the visible portion.

59. A diagnostic device according to any one of claims 56 to 58 wherein the testing portion tapers inwardly from the inlet to the outlet.

60. A diagnostic device according to claim 59 wherein the testing portion tapers along a variable gradient from the inlet to the outlet such that the testing portion tapers inwardly along a first gradient adjacent to the inlet and along a second gradient adjacent to the outlet, the second gradient being steeper than the first gradient.

61. A diagnostic device for detecting a first member of a reporter-analyte pair comprising:
a casing comprising a testing portion having an aperture therein, the testing portion having an inlet for receiving a liquid, biological sample and an outlet for releasing the liquid, biological sample;
a porous membrane element having a detection portion on which a second member of the reporter-analyte pair is immobilised, the outlet being in liquid communication with the detection portion via a detection flow path;
the outlet of the testing portion contacting the membrane element and defining a visible portion of the membrane element, the visible portion of the membrane element comprising the detection portion,
wherein the detection portion is non-planar.

62. A diagnostic device according to claim 61 wherein the detection portion is concave.

63. A diagnostic device according to claim 61 wherein the detection portion is convex.

64. The diagnostic device according to any one of claims 51 to 63, wherein the second member of the reporter-analyte pair comprises a biological antigen and the first member of the reporter-analyte pair comprises an antibody specific for the biological antigen.

65. The diagnostic device according to any one of claims 45, 46 or 64, wherein the biological antigen comprises a spike protein, or a fragment thereof, of COVID-19.

66. The diagnostic device according to claim 65, wherein the spike protein comprises a COVID-19 S1 spike protein or a fragment thereof.

67. The diagnostic device according to claim 64, wherein the biological antigen is a plurality of biological antigens and comprises a first and a second biological antigen and optionally a third biological antigen,
wherein the first biological antigen comprises the COVID-19 S1 spike protein, or a fragment thereof, the second biological antigen comprises the COVID-19 S2 spike protein, or a fragment thereof, and the optional third biological antigen comprises the COVID-19 nucleoprotein, or a fragment thereof.


SEQUENCE LISTING (7 sequences)

SEQ ID NO: 1 - protein, 695 aa, Human coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAY

SEQ ID NO: 2 - protein, 1273 aa, Human coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 223 aa, Human coronavirus
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 4 - protein, 685 aa, Human coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 5 - protein, 588 aa, Human coronavirus
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - protein, 458 aa, Human coronavirus
SFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 7 - protein, 419 aa, Human coronavirus
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA